(19) United States
(12) Patent Application Publication
(10) Pub. No.: US 2021/0379068 A1
(43) Pub. Date: Dec. 9, 2021

(54) METHODS AND MATERIALS FOR TREATING NEUROPSYCHIATRIC DISORDERS

(71) Applicants: Mayo Foundation for Medical Education and Research, Rochester, MN (US); University of Newcastle Upon Tyne, Newcastle upon Tyne (GB)

(72) Inventors: James L. Kirkland, Rochester, MN (US); Mikolaj B. Ogrodnik, Rochester, MN (US); Tamar Tchkonia, Rochester, MN (US); Diana Jurk, Rochester, MN (US); Thomas von Zglinicki, Whitley Bay (GB)

(21) Appl. No.: 17/288,258

(22) PCT Filed: Dec. 18, 2019
(86) PCT No.: PCT/US2019/067147

§ 371 Date: Apr. 23, 2021

Related U.S. Application Data
(60) Provisional application No. 62/782,995, filed on Dec. 20, 2018.

(51) Int. Cl. A61K 31/506; A61K 31/352; A61P 25/28

(52) U.S. Cl. CPC A61K 31/506; A61P 25/28; A61K 31/352

(57) ABSTRACT

This document provides methods and materials for treating obesity-induced neuropsychiatric disorders. For example, one or more senotherapeutic agents can be administered to a mammal having, or at risk of developing, an obesity-induced neuropsychiatric disorder (e.g., obesity-induced anxiety) to treat the mammal.

### CROSS-REFERENCE TO RELATED APPLICATIONS

This application claims priority to U.S. Application Ser. No. 62/782,995, filed on Dec. 20, 2018. The disclosure of the prior application is considered part of the disclosure of this application, and is incorporated in its entirety into this application.

### BACKGROUND

### 1. Technical Field

This document relates to methods and materials for treating obesity-induced neuropsychiatric disorders. For example, one or more senotherapeutic agents can be administered to a mammal having, or at risk of developing, an obesity-induced neuropsychiatric disorder (e.g., obesity-induced anxiety) to treat the mammal.

### 2. Background Information

Obesity can be associated with a range of neurodegenerative and psychiatric disorders, including anxiety and depression in some cases (Gariepy et al., Int J Obes (Lond), 34:407-419 (2010); Hryhorczuk et al., Front Neurosci, 7:177 (2013); Stunkard and Wadden, Am J Clin Nutr, 55:524S-532S (1992)). Anxiety is a behavioral trait in some obese patients (Gariepy et al., Int J Obes (Lond), 34:407-419 (2010)), affecting 40% more obese patients and non-obese patients. Increased anxiety-like behavior also was reported in rodents genetically predisposed to develop obesity, e.g., db/db mice (Dinel et al., PLoS One 6:e24325 (2011)) and in high fat (HF) diet-induced obesity (Heyward et al., Neurobiol Learn Mem 98:25-32 (2012); and Mizunoya et al., Springerplus 2:165 (2013)). Processes such as inflammation (Capuron and Miller, Pharmacol Ther 130:226-238 (2011); and Lasselin and Capuron, 2014), altered hormone signaling (Ulrich-Lai and Ryan, Cell Metab 19:910-925 (2014)), and stem cell dysfunction (Anacker and Hen, Nat Rev Neurosci 18:335-346 (2017); and Gao et al., Neurochem Int 106:24-36 (2017)) have been speculated to underlie obesity-related anxiety, but the underlying mechanisms have not been identified.

### SUMMARY

This document provides methods and materials related to treating obesity-induced neuropsychiatric disorders. For example, this document provides methods and materials for using one or more senotherapeutic agents to treat a mammal having, or at risk of developing, an obesity-induced neuropsychiatric disorder (e.g., obesity-induced anxiety). In some cases, a mammal having, or at risk of developing, obesity-induced anxiety can be treated with a composition including one or more senotherapeutic agents (e.g., dasatinib and/or quercetin) to reduce or eliminate one or more symptoms of obesity-induced anxiety (e.g., anxiety-like behavior). In some cases, a mammal having, or at risk of developing, obesity-induced anxiety can be treated with a composition including one or more senotherapeutic agents to restore neurogenesis within the mammal.

As demonstrated herein, obesity can result in the accumulation of senescent glial cells in proximity to the lateral ventricle (LV), a region in which adult neurogenesis occurs, and these senescent glial cells can exhibit an accumulation of lipids in senescence (ALISE; e.g., excessive fat accumulation). Also as demonstrated herein, reducing the level of cells with an ALISE phenotype from obese mammals (e.g., high fat-fed and leptin receptor-deficient (db/db) obese mice) can restore neurogenesis and alleviated anxiety-related behavior. The ability to decrease the number of senescent glial cells in the LV of an obese mammal (e.g., by administering one or more senotherapeutic agents to the mammal) can be used to treat the mammal having an obesity-induced neuropsychiatric disorder such as anxiety and depression.

In general, one aspect of this document features methods for treating an obesity-induced neuropsychiatric disorder. The methods can include, or consist essentially of, administering a composition including a senolytic agent to a mammal identified as having an obesity-induced neuropsychiatric disorder. The mammal can be a human. The obesity-induced neuropsychiatric disorder can be obesity-induced anxiety. The obesity-induced neuropsychiatric disorder can be obesity-induced depression. The composition can be effective to clear senescent cells from within the brain of the mammal. The senescent cells can include an ALISE phenotype. The senescent cells can be cleared from in proximity to the lateral ventricle of the brain of the mammal. The composition can be effective to decrease a level of one or more senescence-associated secretory phenotype (SASP) factor polypeptides in the mammal.

In another aspect, this document features methods for increasing neurogenesis. The methods can include, or consist essentially of, administering a composition including a senolytic agent to a mammal identified as having an obesity-induced neuropsychiatric disorder under conditions wherein neurogenesis within the mammal is increased. The mammal can be a human. The obesity-induced neuropsychiatric disorder can be obesity-induced anxiety. The obesity-induced neuropsychiatric disorder can be obesity-induced depression. The neurogenesis can be increased in the brain of the mammal. For example, the neurogenesis can be increased in the subventricular zone of the brain of the mammal. For example, the neurogenesis can be increased in the olfactory bulbs of the mammal. The composition can be effective to decrease a level of one or more SASP factor polypeptides in the mammal.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 shows that obese mice exhibit anxiety-like behavior that is not directly related to an increase in body mass. Behavioral changes were tested in the Open Field (OF) chamber. Dark rectangle marks the central area (25% of total area). (A) Representative movement traces (red lines) for chow- and HF diet (HFD)-fed mice at 10 months of age (baseline). Parameters recorded and analyzed in OF: (B) distance travelled in the central area (as a function of total distance travelled) and (C) entries into the central area. No significant correlations (linear regression) were found in both chow or HFD animals between (D) body mass and the normalized distance mice travelled in the central area and (E) the number of entries into the central area. (F) Representative heat maps of time mice spent in the elevated plus maze (EPM) for chow and HFD mice. Closed arms of the maze are indicated by pink brackets. (G) The frequency of head pokes into open arms and (H) the time mice spent with their head in open arms are significantly decreased in HFD- when compared to chow-fed mice. No correlations (linear regression) were found between body mass and elevated plus maze test parameters in chow and HFD mice: (I) frequency of head pokes into open arms and (J) time spent in open arms. Data are from n=26-30 mice per group, Mean±SEM plotted. * P≤0.05 and ** P≤0.001.

FIG. 2 shows that linear regression analysis revealed no significant correlations between anxiety-like behavior markers, body weight, and % of fat in obese mice. (A) Body mass and (B) normalized body fat in chow- and HFD mice. (C) The total distance travelled and (D) total time spent in the central zone of open field in chow- and HFD mice. Linear regression analysis between body mass (E), total distance travelled in open field, and (F) total time spent in the central zone of the open field in lean and obese mice. Linear regression analysis between % of body fat and anxiety-like behavior parameters in the open field test: (G) normalized distance travelled in the central area, (H) entries to the central zone, (I) total distance travelled during the test, and (J) time spent in the central area. Body fat of chow- and HFD mice was correlated to parameters of anxiety-like behavior measured in the elevated plus maze test: (K) frequency of head pokes into the open arms and (L) time of head spend in open arms. Data are from: n=26-30 mice per group, Mean±SEM plotted. * P≤0.05 and ** P≤0.001.

FIG. 3 shows pharmacogenetic and pharmacologic clearance of senescent cells from obese mice alleviates obesity-related behavioral changes. (A) Eight month-old C57Bl/6^{(INK-ATTAC)} male mice were split into four groups and assigned to chow (n=24) or high fat (HF, n=30) diet and treated at 10 months of age with vehicle (n=12 for chow and n=15 for HF) or AP20187 (n=12 for chow and n=15 for HF) until 13 months of age. (B) Open field testing: Representative movement traces (red lines) of lean and obese INK-ATTAC mice treated with/without AP20187 (AP) indicates that anxiety-like behavior of HFD mice can be alleviated by AP treatment as measured by (C) normalized distance travelled in the central area of the open field box and (D) increased frequency of entries into the central area. Alleviation of anxiety-like behavior of HFD mice was also observed by elevated plus maze test. (E) Representative heat-map images of time mice spent in open and closed sections of the elevated plus maze. Parameters were registered by EthoVision software. (F) Frequency of head pokes into the open arms and (G) time mice spent with their heads in open area, was significantly reduced with HFD and increased with AP. (H) Representative movement traces (red lines) of db/db and heterozygous control mice with or without treatment with senolytic cocktail dasatinib+quercetin (D+Q) in the open field test during a 30 minute trial. Data indicates an improvement of anxiety-like behavior of db/db mice upon D+Q treatment determined by open field test parameters: (I) normalized distance travelled in the middle area and (J) frequency of entries into the middle area of the open field box. In the open field test, INK-ATTAC;db/db mice (K) displayed a significant difference in the distance travelled in the central area after treatment with AP20187 when compared to vehicle, but (L) the number of entries into the central area was not significantly increased after treatment with AP. Data are from n=12-15 mice per group for A-G; n=8-12 mice per group for H-J; n=5-9 mice per group for K-L. Mean±SEM plotted. * P≤0.05 and ** P≤0.001.

FIG. 4 shows additional phenotypic and molecular features of AP20187-treated INK-ATTAC mice. (A) Body mass measurements of mice on chow and HF diet before and after last treatment with AP20187 (AP) shows no change in body weight over time. Additional parameters from the open field test: (B) distance in the middle area as a function of the total distance travelled and to the baseline (measurements before the treatment) and (C) total distance travelled. Normalized to baseline parameters of elevated plus maze testing: (D) head pokes toward the open arms and (E) time spent with the head in the open area of the maze. (F, G) Short-term memory was not affected by obesity or AP treatment as determined by lack of substantial changes in Stone's maze parameters: (F) time needed to finish the maze and (G) frequency of errors. C57Bl6 wild-type mice were used to test off-target effects of AP. (H) Before treatment mice under HF diet showed anxiety-like behavior (measured by distance travelled in the center of the open field box). (I) Treatment of C57Bl6 mice under HF diet with AP showed no difference in behavior between treated and un-treated mice. Db/db and heterozygous lean (db/+) mice were given 2 months of D+Q treatment for 5 days every 2 weeks starting from the age of 4 months. Db/db mice did not show changes in (J) body mass or (K) body fat within the groups over the course of treatment. (L) Total distance travelled by db/db and db/+ mice in the open field test was not affected by D+Q treatment. 3-month old INK-ATTAC; INK-ATTAC:db/db mice were randomly sorted to AP or vehicle groups and treated for 2 months. No changes in (M) body mass or (N) body composition were observed in AP treated mice. (0) Total distance travelled during 30 minute long open field testing was not affected by AP treatment. (P) Db/db mice were tested for off-target effects of AP. Treatment showed no difference in behavior between treated and un-treated mice. (Q-S) Linear regression analysis between body weight and anxiety-like behavior, measured by distance travelled in the centre of the open field box, showed no association between the two in HF diet INK-ATTAC mice treated with and without AP (Q), in db/db mice treated with and without D+Q and (S) in INK-ATTAC:db/db mice treated with and without AP20187. Data are from n=13-15 mice per group for graphs A-E, n=9-24 mice per group for F-G, n=7-12 mice per group for H, n=9-10 mice per group for graphs I, n=6-8 mice per group for graphs J-L, n=8-9 mice per group for graphs M-N, n=16 mice per group for the graph O, n=11 mice per group for the graph P, n=30-31 mice per group for graphs Q and S and n=23 mice per group for the graph R. Mean±SEM plotted. * P≤0.05 and ** P≤0.001.

FIG. 5 shows that decreasing the amount of senescent cells in obese animals reduces circulating cytokine levels. (A) Quantification of % of senescence-associated beta-galactosidase (SA-β-Gal) positive cells in perigonadal adipose tissue shows increased values in HF diet-fed animals and complete rescue after treatment with AP20187. Senescent markers p16 (measured by RT-PCR) (B) and telomere associated DNA damage foci (TAF) (C) show a similar pattern. (D) Representative images and (E) quantification of SA-β-Gal activity in perigonadal adipose tissue of db/db and db/+ mice shows that % of SA-β-Gal positive cells increase in db/db mice compared to db/+ and are significantly reduced upon treatment with the senolytic cocktail D+Q. (F) Frequencies of TAF-positive cells in perigonadal adipose tissue of db/db increase significantly in comparison to db/+ mice and decrease after D+Q treatment. (G) Cytokine protein expression [fold change] in blood plasma from HFD animals treated with/without AP20187. (H) Cytokine expression [fold change] in blood plasma from db/db animals treated with/without AP20187. Linear regression analysis between anxiety markers and cytokines in blood plasma showed a significant negative correlation between (I) Cxcl-1, (J) G-Csf, (K) Mig and the distance travelled in the central zone of the open field box in HF diet fed mice. Data are from n=6-9 mice per group for A-C and G-H; n=7-12 mice per group for D-F and I-J; n=27 mice per group for K-M. Mean±SEM plotted. * P≤0.05 and ** P≤0.001.

FIG. 6 shows influence of systemic factors on anxiety-like behavior. Quantification of p21 (A) by PCR and the number of DNA damage foci (γ-H2A.X) (B) by IF-staining in perigonadal adipose tissue shows increased values in HF-diet fed animals and but no change after treatment with AP20187. Correlations between anxiety-like phenotype markers and Cxcl-1 in blood plasma of HFD and chow animals (C) in EPM, (D) OF and in db/db and db/db^{+/-} animals (E) EPM. Correlations between (F) Tnf-α, (G) Il-6 and (H) Mcp-1 in blood plasma of HF and chow diet animals and the distance travelled in the central zone of the open field box showed no significant differences. To test the effect of increased or decreased cytokine levels in the blood stream mice were injected with Cxcl-1 or treated with Cxcr1 inhibitor Reparixin (I) Cxcl-1 is significantly increased in blood plasma of C57Bl6 mice injected with Cxcl-1 in comparison to vehicle-treated mice. Mice injected with Cxcl- lost weight (J) but did not experience changes in body fat (K). Distance travelled in the central area of the open field box (L) and head entries in the open arms of the EPM (M) were not different between Cxcl-1 and vehicle treated mice. (N) Mice under high fat diet and treated with Reparixin or vehicle showed an increase in body mass, but did show no alterations in body fat (0). No difference in the open field (P) and EPM (Q) test were observed. (R) Scheme showing experimental setup for transplantation experiments. C57Bl6 mice were divided into 3 groups, injected either with PBS, young or senescent mouse preadipocytes and tested for anxiety-like behavior (OF) and frailty (Rotarod). (S) Rotarod testing showed significant decreased performance in mice injected with senescent cells at 12 weeks but not 2 weeks after injection. All mice showed no anxiety-like phenotype 2 and 6 weeks after cell transplantation when distance travelled (T), entries (U), normalized distance travelled in the central area and total distance travelled (V, W) in the open field box were measured. Data are from n=6-8 mice per group for graphs A-B, mice per group n=27 for graphs C and F-H, mice per group n=36 for graphs D-E, n=10 mice per group for graphs I-M, n=12 mice per group for graphs N-Q and n=6-7 mice per group for the graphs S-W. Mean±SEM plotted. * P≤0.05 and ** P≤0.001.

FIG. 7 shows markers of senescence in the amygdala are reduced after treatment with AP20187. (A) Cdkn2a positive cells were measured by RNA-ISH in the basomedial layer of the amygdala. (B) Representative images showing telomere associated DNA damage foci (TAF), (blue=DAPI, red=telomeres, green=γ-H2A.X, white arrow indicates TAF) in 3 μm thick paraffin embedded brain sections. (C) Mean number of TAF and (D) % of NeuN-pos cells with 2 or more TAF was increased in HFD INK-ATTAC mice and significantly reduced after AP20187 treatment in the basomedial layer of the amygdala. (E) Mean number of TAF and (F) % of NeuN-neg cells with 2 or more TAF was increased in HFD INK-ATTAC mice and significantly reduced after AP20187 treatment in the hypothalamus in close proximity to the 3^{rd} ventricle. Data are from n=5-6 mice per group for graphs B-D; Mean±SEM plotted. * P≤0.05 and ** P≤0.001.

FIG. 8 shows levels of senescent markers are not changed in cortex, cerebellum, or hippocampus of HFD when compared to lean animals. Analysis of p21 (A) and p16 (B) by RT-PCR in different brain regions (cortex, cerebellum, and hippocampus) revealed no significant difference among groups. Quantification of γ-H2A.X foci (C) and telomere associated damage foci (TAF) (D) in neurons in different brain areas showed no significant difference. (E) Mean number of TAF and (F) % of NeuN-pos cells with 2 or more TAF was increased in HFD INK-ATTAC mice and significantly reduced after AP20187 treatment in the hypothalamus in close proximity to the 3^{rd} ventricle. Data are from n=5-8 mice per group for graphs A-D; n=5 for E, F. Mean±SEM plotted. * P≤0.05 and ** P≤0.001.

FIG. 9 shows obesity-related accumulation of lipid droplets in senescent periventricular glia is reduced upon senescent cells clearance. (A) Representative images of Perilipin 2 (Plin2) staining showing accumulation of cells exhibiting build-up of lipid droplets in close proximity to the lateral ventricle (LV) of middle-aged, obese mice compared to their lean littermates. (B) Quantification of frequencies of Plin2⁺ cells in the proximity (up to 250 μm from ependymal cell layer) to the LV in high fat (HF)-fed and lean mice. (C) Representative images showing Plin2⁺ cells co-localizing with markers of microglia (Iba1; top left panel) and astrocytes (vimentin [Vim]; top right panel) but not with neuronal markers (NeuN; bottom panel). (D) Pie chart shows cell-type composition of Plin2⁺ cells [determined after Immunostaining for Plin2 and different cell-type markers (as shown in C)]. (E) Periventricular Plin2⁺ glial cells show increased numbers of senescent-marker telomere associated foci (TAF). Quantification of mean number of TAF per cell in non-neuronal (NeuN_{neg}) Plin⁺ and Plin⁻ cells. (F) Representative Images show the LV of chow (top panel) and HF AP20187-treated mice stained with Plin2, exhibiting reduced lipid droplets. (G) Quantification of cells containing lipid droplets (Plin2⁺) in the periventricular area of lean HF INK-ATTAC mice with or without AP20187 treatment. (H) Quantification of frequencies of NeuN negative, TAF-positive cells in the periventricular region of lean/HF and vehicle or AP20187-treated mice. (I) Representative images showing double staining for CXCL1 (RNA-ISH in red) and Plin2 (green) in periventricular area of HF INK-ATTAC mice. White arrows indicate CXCL1 and Plin2 double positive cells. Cells magnified in panel 4 encircled in red show positive staining for Plin2 and CXCL1, whereas white-bordered cells are not double positive. Quantification of cells positive for Cxcl1 (J) and Il-6 (K) stained by RNA-ISH indicate that Plin⁺ cells display significantly higher expression levels of senescence-associated secretory phenotype (SASP) factors Il-6 and Cxcl1 than Plin⁻ cells. Build-up of fat in periventricular brain region as assessed by Plin2 staining significantly correlates with parameters associated with anxiety-like behavior: (L) % of distance travelled in the central zone (% of central zone) and (M) number of entries into the central zone (entries) in open field testing. Data are from n=5-8 mice per group for B, n=6 mice per group for E, J and K, n=4-8 mice per group for G-H, and n=25 mice per group for L and M. Mean±SEM plotted. *P≤0.05 and ** P≤0.001.

FIG. 10 shows assessment of periventricular fat accumulation and markers of senescence in lean and obese mice. (A) Lipid droplets in periventricular cells visualized by Perilipin 2 (Plin2) staining. The panel on the top right shows a magnified cell and the panel on the bottom right shows magnified lipid droplets visualized by Plin2 staining. (B) Plin2 staining shows accumulation of lipid droplets in cells in close proximity to the 3^{rd} (left panel), the 4^{th} ventricle (middle panel) and periaqueductal grey matter (PAG) (right panel). Bottom left images show merged images of Plin2 and DAPI staining. (C) γ-H2A.X foci were quantified in Plin2⁺ and Plin2⁻, non-neuronal cells in the lateral ventricle. (D) Images showing the ependymal layer in the LV of db/db vehicle (top panel) and db/db D+Q-treated mice stained with Plin2 in red and S100β in green. Areas in white rectangles are magnified on the right and show reduction in size of Plin2 lipid droplets in D+Q treated animals. (E) Quantification of the area containing lipid droplets (Plin2⁺) in the ependymal layer of db/db and db/db^{-/-} mice with or without D+Q treatment. (F) Quantification of frequencies TAF-positive cells in the ependymal layer of db/db and db/+ and vehicle or D+Q-treated mice. Data are from n=6 mice per group for graph C, n=7-12 mice per group for graph E, F. Mean±SEM plotted. * P≤0.05 and ** P≤0.001.

FIG. 11 shows ALISE phenotype drives CCF accumulation and SASP. Similarly to glia in the brains of obese mice, mouse adult fibroblasts (MAFs) show an Accumulation of Lipids In SEnescence (ALISE) phenotype. (A-F) Depletion of lipids from culture media reduces area of lipid droplets in MAFs. (A) ALISE phenotype in MAFs is characterised by increased area of lipid droplets surrounded by Plin2 vesicles. (B) Quantification of Nile Red-positive staining in young (you) and senescent (sen) MAFs. (C-D) Suppression of ALISE phenotype reduces frequencies of Cytoplasmic Chromatin Fragments (CCF) in senescent fibroblasts but (E-F) does not affect number of 53BP1 DNA damage foci. (G-K) Senescent fibroblasts have increased secretion of SASP components including Il-6, KC (Cxcl1), and Ip-10 (Cxcl10) that is alleviated upon suppression of ALISE phenotype. (G) Heat map shows fold change in secretion of SASP components to cell culture media over a 72 hour time-period when compared to young fibroblasts cultured with lipid-containing media. Each square represents a separate biological replicate, MAFs isolated from a different mouse donor. Concentration of SASP components in culture media with and without lipids (ALISE phenotype suppression) for (H) Il-6, (I) Ip-10 (Cxcl10), (J) Vegf, and (K) Kc (Cxcl1). (L) Images show accumulation of lipid droplets in senescent but not young GFAP-positive astrocytes. (M-O) Characterization of senescence in astrocytes. Senescent astrocytes show (M) the ALISE phenotype measured using Nile Red staining, (N) increased frequencies of telomere dysfunction measured as frequency of cells with 1 or more telomere associated DNA damage foci (TAF), and (O) increased frequencies of CCF. Senescence was induced by X-ray irradiation (10 Gy) and established within 14-21 days post-irradiation. Data are from n=3-6 mice per group. Mean±SEM plotted. * P≤0.05 and ** P≤0.001.

FIG. 12 shows an analysis of ALISE phenotype in astrocytes. (A) Suppression of the ALISE phenotype by culturing cells in lipid free media doesn't change bi-nuclearity in MAFs. In astrocytes, the average number of TAF (B), average number of 53BP1 foci (C) and bi-nuclearity (D) increases significantly after induction of senescence. Senescence was induced by X-ray irradiation (10 Gy) and established within 14-21 days post-irradiation. Data are from n=3 mice. Mean±SEM plotted. * P≤0.05 and ** P≤0.001.

FIG. 13 shows clearance of senescent cells partially reverses the neural progenitor cell depletion induced by obesity. (A) Each hemisphere of INK-ATTAC lean and high fat (HF) mouse brain was either dissociated into a single-cell suspension or processed for IHC/IF. Dissociated brain cells were labelled with metal-conjugated antibodies and processed for Cytometry by Time of Flight (CyTOF). (B, D) Spanning-tree Progression Analysis of Density-normalized Events (SPADE) was performed on brain cell populations identified by markers shown on the micrographs. Heat map shows the intensity of antibody signal and the size of each spot is determined by the number of cells within this population. (C) CyTOF shows differences in brain cell populations of INK-ATTAC chow and HF mice, which were treated with vehicle or AP20187. Frequencies of cells expressing markers (E) doublecortin (Dcx), (F) CD133 and (G) Nestin were quantified. (H) Representative images of doublecortin (Dcx) staining in the olfactory bulb of chow- and HFD mice treated with vehicle or AP20187. White boxes show magnified regions. (I) Quantitative analysis of Dcx-positive area of the granular layer in the olfactory lobe of lean and obese INK-ATTAC mice and (J) correlation between area occupied by Dcx⁺ cells in the granular layer of the olfactory bulb and frequencies of Dcx⁺ cells from the whole brain measured by CyTOF. Correlations (linear regression analysis) between frequencies of periventricular glia exhibiting accumulation of lipid droplets and frequencies of cells expressing markers of neurogenesis and ependymal cells (determined by CyTOF): (K) Nestin and (L) Dcx. Correlations (linear regression analysis) between distance travelled in the central zone and frequencies of cells expressing (M) Nestin and (N) Dcx (determined by CyTOF). Data are from n=5-9 mice per group for C-G; n=2-6 mice per group for I; n=11 mice per group for J; n=29 mice per group for K-N. Mean±SEM plotted. * P≤0.05 and ** P≤0.001.

FIG. 14 shows association between adult neurogenesis and periventricular lipid accumulation. (A) Cells displaying perilipin 2 (Plin2)-positive lipid droplets were found in close proximity to doublecortin (Dcx)-positive cells. Yellow box marks magnified region shown on the right. Characterization by Cytometry by Time of Flight (CyTOF) of different cell-types in the brain of lean and obese INK-ATTAC mice with or without AP treatment. Quantification of markers of oligodentrocytes: (B) 2′,3′-cyclic-nucleotide 3′-phosphodiesterase (CNPase), (C) oligodendrocyte specific protein (OSP), (D) double positive cells for CNPase and OSP; markers of microglia: (E) CD11b and (F) CD45⁻/CD11b⁺; markers of astrocytes: (G) astrocyte cell surface antigen-2 (ACSA-2), (H) glial fibrillary acidic protein (Gfap), and (I) double-positive for ACSA-2 and Gfap; (J) marker of astrocytes, epithelial cells, pericytes, and ependymal cells vimentin (Vim); markers of endothelial cells and pericytes: (K) CD146 and (L) CD31; and a (M) marker of neurons, NeuN. One hemisphere of each db/db vehicle or D+Q treated mouse brain was dissociated into a single-cell suspension and labelled with metal-conjugated antibodies and processed for (CyTOF). Frequencies of cells expressing markers (N) doublecortin (Dcx), (0) CD133 and (P) Nestin were quantified. (Q) GFAP staining (blue is DAPI) in the cerebral cortex and (R) its quantification (in the indicated areas). (S) Representative images of EdU staining in the preventricular area of chow- and HF diet-fed mice treated with vehicle or AP20187. Yellow boxes show magnified regions. (T) Quantitative analysis of EdU-positive cells per image in lean and obese INK-ATTAC mice with or without AP treatment. (U) A representative image of immunofluorescent staining for DCX in the dentate gyrus (DG) of the hippocampus. (V) Frequencies of DCX-positive cells in the DG and (W) total amount of EdU-positive cells in the hippocampus per hemisphere in lean and HFD INK-ATTAC animals with and without AP treatment. Data are from n=6-9 mice per group for graphs B-M, n=8 mice per group for graphs N-P, n=5-8 mice per group for the graph R, n=3 mice per group for the graph T, n=5-8 mice per group for the graph V, n=6 mice per group for the graph W. Mean+/−SEM plotted. * P≤0.05 and ** P≤0.001.

### DETAILED DESCRIPTION

This document provides methods and materials related to treating obesity-induced neuropsychiatric disorders. For example, this document provides methods and materials for using one or more senotherapeutic agents (e.g., dasatinib and/or quercetin) to treat a mammal having an obesity-induced neuropsychiatric disorder (e.g., obesity-induced anxiety). In some cases, one or more senotherapeutic agents (e.g., dasatinib and/or quercetin) can be used as described herein to treat a mammal at risk of developing an obesity-induced neuropsychiatric disorder (e.g., obesity-induced anxiety).

In some cases, a mammal having, or at risk of developing, an obesity-induced neuropsychiatric disorder can be treated with a composition including one or more senotherapeutic agents (e.g., dasatinib and/or quercetin) to alleviate (e.g., to reduce or eliminate) one or more (e.g., one, two, three, four, five, or more) symptoms of the obesity-induced neuropsychiatric disorder. An obesity-induced neuropsychiatric disorder can be any type of obesity-induced neuropsychiatric disorder. Examples of obesity-induced neuropsychiatric disorder include, without limitation, obesity-induced anxiety, obesity-induced depression, obesity-induced fearfulness, obesity-related suicide, and obesity-induced stress. A symptom of an obesity-induced neuropsychiatric disorder can be any appropriate symptom. For example, examples of symptoms of obesity-induced anxiety include, without limitation, anxiety-related behaviors such as feeling nervous, feeling restless, feeling tense, feeling stressed, having a sense of impending danger, increased heart rate, hyperventilation, sweating, trembling, feeling weak or tired, trouble concentrating or thinking about anything other than the present worry, having trouble sleeping, and gastrointestinal problems. Each of these symptoms of obesity-induced anxiety can be identified, staged, and/or monitored using clinical techniques as described elsewhere (see, e.g., Practice Guidelines for Psychiatric Evaluation of Adults, Third Edition, American psychiatric association, 2016; Lykouras et al., Psychiatriki 22:307-13 (2011); and Locke et al., Am Fam Physician 91:617-24 (2015)). For example, examples of symptoms of obesity-induced depression include, without limitation, feelings of sadness, feelings of tearfulness, feelings of hopelessness, feelings of worthlessness, angry outbursts, irritability or frustration, loss of interest or pleasure normal activities hobbies or sports, sleep disturbances, lack of energy, fixating on past failures or self-blame, frequent or recurrent thoughts of death, suicidal thoughts, suicide attempts, and unexplained physical problems such as back pain or headaches. Each of these symptoms of obesity-induced depression can be identified, staged, and/or monitored using clinical techniques as described elsewhere (see, e.g., Practice Guidelines for Psychiatric Evaluation of Adults, Third Edition, American psychiatric association, 2016; and Clinical Practice Guidelines, American Psychiatric Association, available at psychiatry.org/psychiatrists/practice/clinical-practice-guidelines) In some cases, administering one or more senotherapeutic agents to a mammal having, or at risk of developing, an obesity-induced neuropsychiatric disorder can be effective to alleviate one or more anxiety-related behaviors in the mammal.

In some cases, a mammal having, or at risk of developing, an obesity-induced neuropsychiatric disorder (e.g., obesity-induced anxiety) can be treated with a composition including one or more senotherapeutic agents (e.g., dasatinib and/or quercetin) to clear one or more senescent cells from within the mammal. A senescent cell can be any type of cell. In some cases, a senescent cell can exhibit excessive fat accumulation (e.g., can have an ALISE phenotype). Examples of senescent cells that can be cleared as described herein include, without limitation, a senescent glial cell, an ependymal cell, a neural progenitor cell, a neuron, and an endothelial cell. A senescent cell can be cleared from any location within the mammal. In some cases, a senescent cell can be cleared from the brain of a mammal. Examples of locations from which a senescent cell cleared include, without limitation, in proximity to the LV of the brain of the mammal, in the LV of the brain of the mammal, in proximity to the subventricular zone (SVZ) of the brain of the mammal, in the SVZ of the brain of the mammal, and cerebral blood vessels. A location in proximity to the LV of the brain of a human can be the region within about 10 mm (within about 9 mm, within about 8 mm, within about 7 mm, within about 6 mm, within about 5 mm, within about 4 mm, within about 3 mm, within about 2 mm, or within about 1 mm) of the LV. A location in proximity to the SVZ of the brain of a human can be the region within about 10 mm (within about 9 mm, within about 8 mm, within about 7 mm, within about 6 mm, within about 5 mm, within about 4 mm, within about 3 mm, within about 2 mm, or within about 1 mm) of the SVZ. In some cases, administering one or more senotherapeutic agents to a mammal having, or at risk of developing, an obesity-induced neuropsychiatric disorder can be effective to clear one or more senescent cells having an ALISE phenotype from a location in proximity to the LV of the brain of the mammal.

In some cases, a mammal having, or at risk of developing, an obesity-induced neuropsychiatric disorder (e.g., obesity-induced anxiety) can be treated with a composition including one or more senotherapeutic agents (e.g., dasatinib and/or quercetin) to increase (e.g., restore) neurogenesis within the mammal. In some cases, a mammal having, or at risk of developing, an obesity-induced neuropsychiatric disorder (e.g., obesity-induced anxiety) can be treated with a composition including one or more senotherapeutic agents (e.g., dasatinib and/or quercetin) to alleviate (e.g., to reduce or eliminate) obesity-related impairment of neurogenesis in the mammal. Neurogenesis of any appropriate type of cell can be increased. Examples of cells for which neurogenesis can be increased as described herein include, without limitation, neuronal precursor cells, immature neurons, ependymal cells, and developing neurons. Neurogenesis can be increased in any location within the mammal. Examples of locations in which a neurogenesis can be increased include, without limitation, in the SVZ of the brain of the mammal, and in the olfactory bulbs of the mammal. In some cases, administering one or more senotherapeutic agents to a mammal having, or at risk of developing, an obesity-induced neuropsychiatric disorder can be effective to restore neurogenesis in the mammal.

In some cases, a mammal having, or at risk of developing, an obesity-induced neuropsychiatric disorder (e.g., obesity-induced anxiety) can be treated with a composition including one or more senotherapeutic agents (e.g., dasatinib and/or quercetin) to alleviate (e.g., to reduce or eliminate) inflammation in the mammal. A level (e.g., a systemic level) of any appropriate inflammatory factor (e.g., cytokines, chemokines, and matrix proteases) can be altered (e.g., increased or decreased) to alleviate inflammation in a mammal having, or at risk of developing, an obesity-induced neuropsychiatric disorder. In cases where an inflammatory factor is a pro-inflammatory factor (e.g., SASP factor polypeptides such as G-Csf, Il-1α and Il-1β, Kc/Cxcl1, Mcp-1, Mig, Il-6, Tnf-α; and IL-8) the pro-inflammatory factor can be decreased. In cases where an inflammatory factor is an anti-inflammatory factor, the anti-inflammatory factor can be increased. Inflammation at any appropriate location within the mammal can be alleviated. Examples of locations from which inflammation can be alleviated as described herein include, without limitation, the brain, blood vessels, adipose tissue, the lungs, kidneys, the liver, bone, bone marrow, and skin. In some cases, a systemic inflammatory factor (e.g., systemic SASP factor polypeptides) can cross the blood-brain barrier to alleviate brain inflammation. In some cases, administering one or more senotherapeutic agents to a mammal having, or at risk of developing, an obesity-induced neuropsychiatric disorder can be effective to alleviate brain inflammation within the mammal.

In some cases, when a mammal having, or at risk of developing, an obesity-induced neuropsychiatric disorder (e.g., obesity-induced anxiety) is treated with a composition including one or more senotherapeutic agents (e.g., dasatinib and/or quercetin), the mammal's body weight is not affected (e.g., is not altered).

In some cases, when a mammal having, or at risk of developing, an obesity-induced neuropsychiatric disorder (e.g., obesity-induced anxiety) is treated with a composition including one or more senotherapeutic agents (e.g., dasatinib and/or quercetin), the mammal's body composition is not affected (e.g., is not altered).

In some cases, when a mammal having, or at risk of developing, an obesity-induced neuropsychiatric disorder (e.g., obesity-induced anxiety) is treated with a composition including one or more senotherapeutic agents (e.g., dasatinib and/or quercetin), the mammal's activity is not affected (e.g., is not altered).

When treating a mammal having, or at risk of developing, an obesity-induced neuropsychiatric disorder (e.g., obesity-induced anxiety) as described herein (e.g., by administering one or more senotherapeutic agents such as dasatinib and/or quercetin), the mammal can be any appropriate mammal. In some cases, a mammal can be an obese mammal (e.g., a mammal that is overweight). Examples of mammals that can be treated using a composition containing one or more senotherapeutic agents as described herein include, without limitation, humans, non-human primates such as monkeys, dogs, cats, horses, cows, pigs, sheep, mice, and rats. In some cases, a composition containing one or more senotherapeutic agents can be administered to a human having an obesity-induced neuropsychiatric disorder to treat the human. In some cases, a composition containing one or more senotherapeutic agents can be administered to a human at risk of developing an obesity-induced neuropsychiatric disorder to slow the onset or progression of an obesity-induced neuropsychiatric disorder within the human.

In some cases, the methods described herein also can include identifying a mammal as having, or as being at risk of developing, an obesity-induced neuropsychiatric disorder (e.g., obesity-induced anxiety). Examples of methods for identifying a mammal as having, or as being at risk of developing, an obesity-induced neuropsychiatric disorder include, without limitation, psychological evaluation, physical examination, and/or laboratory tests such as stress hormone levels. Once identified as having, or as being at risk of developing, an obesity-induced neuropsychiatric disorder, a mammal can be administered or instructed to self-administer one or more senotherapeutic agents (e.g., dasatinib and/or quercetin).

A composition containing one or more (e.g., one, two, three, four, five, or more) senotherapeutic agents can include any appropriate senotherapeutic agent(s). A senotherapeutic agent can be any type of molecule (e.g., small molecules or polypeptides). In some cases, a senotherapeutic agent can be a senolytic agent (i.e., an agent having the ability to induce cell death in senescent cells). In some cases, a senotherapeutic agent can be a senomorphic agent (i.e., an agent having the ability to suppress senescent phenotypes without cell killing). Examples of senotherapeutic agents that can be used as described herein (e.g., to treat a mammal having, or at risk of developing, an obesity-induced neuropsychiatric disorder such as obesity-induced anxiety) can include, without limitation, dasatinib, quercetin, navitoclax, A1331852, A1155463, fisetin, luteolin, geldanamycin, tanespimycin, alvespimycin, piperlongumine, panobinostat, FOX04-related peptides, nutlin3a, ruxolitinib, metformin, and rapamycin.

In some cases, a composition containing one or more (e.g., one, two, three, four, five, or more) senotherapeutic agents (e.g., dasatinib and/or quercetin) can include the one or more senotherapeutic agent(s) as the sole active ingredient(s) in the composition that is effective to treat an obesity-induced neuropsychiatric disorder (e.g., obesity-induced anxiety). In some cases, a composition containing one senotherapeutic agent (e.g., fisetin) can include that one senotherapeutic agent as the sole active ingredient in the composition that is effective to treat an obesity-induced neuropsychiatric disorder (e.g., obesity-induced anxiety).

In some cases, a composition containing one or more (e.g., one, two, three, four, five, or more) senotherapeutic agents (e.g., dasatinib and/or quercetin) can include one or more (e.g., one, two, three, four, five, or more) additional active agents (e.g., therapeutic agents) in the composition that are effective to treat an obesity-induced neuropsychiatric disorder (e.g., obesity-induced anxiety).

In some cases, a mammal having, or at risk of developing, an obesity-induced neuropsychiatric disorder (e.g., obesity-induced anxiety) being treated as described herein (e.g., by administering one or more senotherapeutic agents such as dasatinib and/or quercetin) also can be treated with one or more (e.g., one, two, three, four, five, or more) additional therapeutic agents. A therapeutic agent used in combination with one or more senotherapeutic agents described herein can be any appropriate therapeutic agent. Examples of therapeutic agents that can be used in combination with one or more senotherapeutic agents described herein include, without limitation, benzodiazepines (e.g., alprazolams such as XANAX™, chlordiazepoxides such as LIBRIUIM®, clonazepams such as KLONOPIN®, diazepams such as VALIUM®, and lorazepams such as ATIVAN®), buspirone, and antidepressants including selective serotonin reuptake inhibitors (SSRIs; e.g., escitaloprams such as LEXAPRO, fluoxetines such as PROZAC®, paroxetines such as PAXIL®, and sertralines such as ZOLOFT®). In some cases, the one or more additional therapeutic agents can be administered together with the one or more senotherapeutic agents (e.g., in a composition containing one or more senotherapeutic agents and containing one or more additional therapeutic agents). In some cases, the one or more (e.g., one, two, three, four, five, or more) additional therapeutic agents can be administered independent of the one or more senotherapeutic agents. When the one or more additional therapeutic agents are administered independent of the one or more senotherapeutic agents, the one or more senotherapeutic agents can be administered first, and the one or more additional therapeutic agents administered second, or vice versa.

In some cases, a composition containing one or more senotherapeutic agents (e.g., dasatinib and/or quercetin) can be formulated into a pharmaceutically acceptable composition for administration to a mammal having, or at risk of developing, an obesity-induced neuropsychiatric disorder (e.g., obesity-induced anxiety). For example, one or more senotherapeutic agents can be formulated together with one or more pharmaceutically acceptable carriers (additives) and/or diluents. Pharmaceutically acceptable carriers, fillers, and vehicles that can be used in a pharmaceutical composition described herein include, without limitation, saline, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol (PEG; e.g., PEG400), sodium carboxymethylcellulose, polyacrylates, waxes, polyethylene-polyoxypropylene-block polymers, and wool fat.

In some cases, when a composition containing one or more senotherapeutic agents (e.g., dasatinib and/or quercetin) is administered to a mammal having, or at risk of developing, an obesity-induced neuropsychiatric disorder (e.g., obesity-induced anxiety), the composition can be designed for oral or parenteral (including subcutaneous, intramuscular, intravenous, and intradermal) administration to the mammal. Compositions suitable for oral administration include, without limitation, liquids, tablets, capsules, pills, powders, gels, and granules. Compositions suitable for parenteral administration include, without limitation, aqueous and non-aqueous sterile injection solutions that can contain anti-oxidants, buffers, bacteriostats, and solutes that render the formulation isotonic with the blood of the intended recipient.

A composition containing one or more senotherapeutic agents (e.g., dasatinib and/or quercetin) can be administered to a mammal having, or at risk of developing, an obesity-induced neuropsychiatric disorder (e.g., obesity-induced anxiety) in any appropriate amount (e.g., dose). Effective amounts can vary depending on the route of administration, the age and general health condition of the subject, excipient usage, the possibility of co-usage with other therapeutic treatments such as use of other agents, and the judgment of the treating physician. An effective amount of a composition containing one or more senotherapeutic agents can be any amount that can treat a mammal having, or at risk of developing, an obesity-induced neuropsychiatric disorder without producing significant toxicity to the mammal. For example, an effective amount of dasatinib (D) can be from about 1 milligram per kilogram body weight (mg/kg) to about 20 mg/kg (e.g., about 5 mg/kg). For example, an effective amount of quercetin (Q) can be from about 10 mg/kg to about 200 mg/kg (e.g., about 50 mg/kg). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the obesity-induced neuropsychiatric disorder in the mammal being treated may require an increase or decrease in the actual effective amount of senotherapeutic agent(s) administered.

A composition containing one or more senotherapeutic agents (e.g., dasatinib and/or quercetin) can be administered to a mammal having, or at risk of developing, an obesity-induced neuropsychiatric disorder (e.g., obesity-induced anxiety) in any appropriate frequency. The frequency of administration can be any frequency that can treat a mammal having, or at risk of developing, an obesity-induced neuropsychiatric disorder without producing significant toxicity to the mammal. For example, the frequency of administration can be from about twice a day to about once every 6 months, from about once a day to about once a week, or from about once a week to about once every 6 months. In some cases, a composition containing one or more senotherapeutic agents can be administered once a day. The frequency of administration can remain constant or can be variable during the duration of treatment. As with the effective amount, various factors can influence the actual frequency of administration used for a particular application. For example, the effective amount, duration of treatment, use of multiple treatment agents, and route of administration may require an increase or decrease in administration frequency.

A composition containing one or more senotherapeutic agents (e.g., dasatinib and/or quercetin) can be administered to a mammal having, or at risk of developing, an obesity-induced neuropsychiatric disorder (e.g., obesity-induced anxiety) for any appropriate duration. An effective duration for administering or using a composition containing one or more senotherapeutic agents can be any duration that can treat a mammal having, or at risk of developing, an obesity-induced neuropsychiatric disorder without producing significant toxicity to the mammal. For example, the effective duration can vary from several days, to several weeks, to several months, or to a lifetime. In some cases, the effective duration can range in duration from about several months to about 10 years. Multiple factors can influence the actual effective duration used for a particular treatment. For example, an effective duration can vary with the frequency of administration, effective amount, use of multiple treatment agents, and route of administration.

In certain instances, a course of treatment can be monitored. In some cases, methods described herein also can include monitoring the severity of an obesity-induced neuropsychiatric disorder (e.g., obesity-induced anxiety) in a mammal. Any appropriate method can be used to monitor the severity of an obesity-induced neuropsychiatric disorder in a mammal. In some cases, methods described herein also can include monitoring a mammal being treated as described herein for toxicity. The level of toxicity, if any, can be determined by assessing a mammal's clinical signs and symptoms before and after administering a known amount of a particular composition. It is noted that the effective amount of a particular composition administered to a mammal can be adjusted according to a desired outcome as well as the mammal's response and level of toxicity.

The invention will be further described in the following examples, which do not limit the scope of the invention described in the claims.

### EXAMPLES

### Example 1: Obesity Induced Cellular Senescence Drives Anxiety-Like Behavior Via Impaired Neurogenesis

This example shows that during obesity, glial cells show increased markers of cellular senescence in the periventricular region of the lateral ventricle (LV), a region in close proximity to the neurogenic niche. Senescent glial cells in obese mice show excessive fat accumulation, a phenotype termed accumulation of lipids in senescence (ALISE). Importantly, specific clearance of senescent cells alleviates the obesity-related impairment in adult neurogenesis, and decreases obesity-induced anxiety-like behavior. This work suggests that targeting senescent cells can be used as a therapeutic avenue for treating obesity-induced anxiety.

### Results

### Obese Mice Show Increased Anxiety-Like Behavior not Related to Body Mass

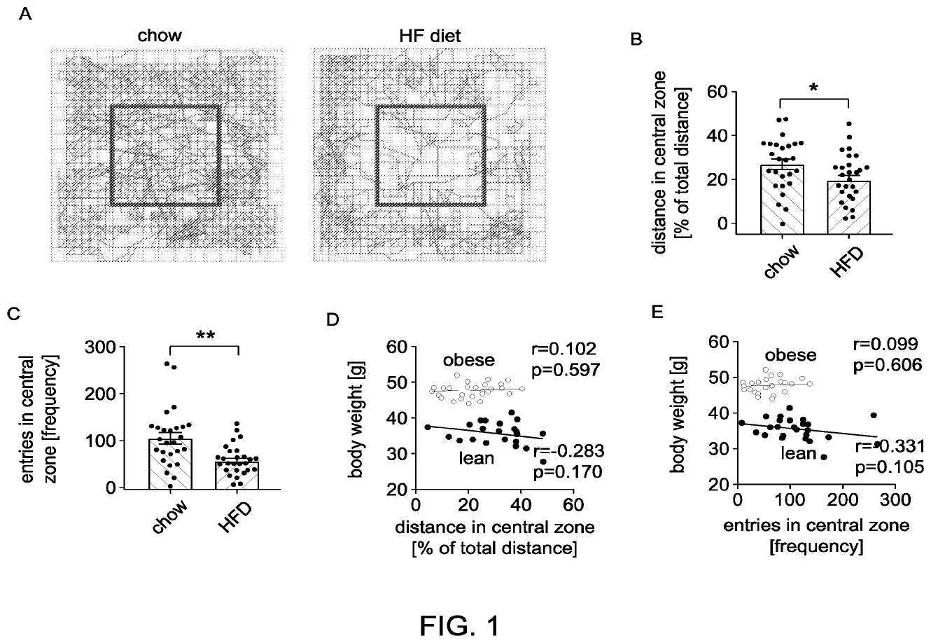

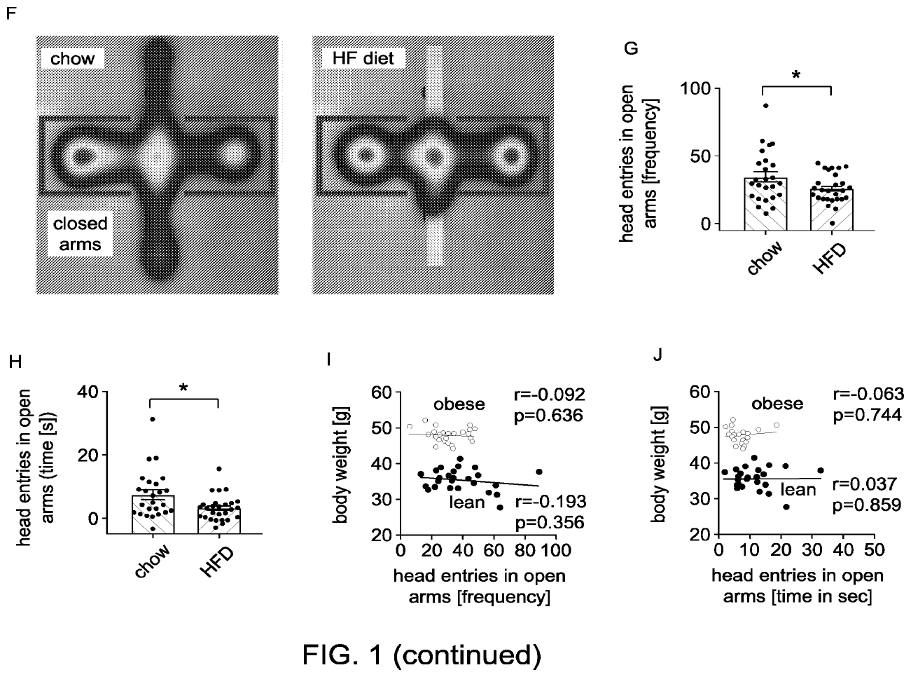

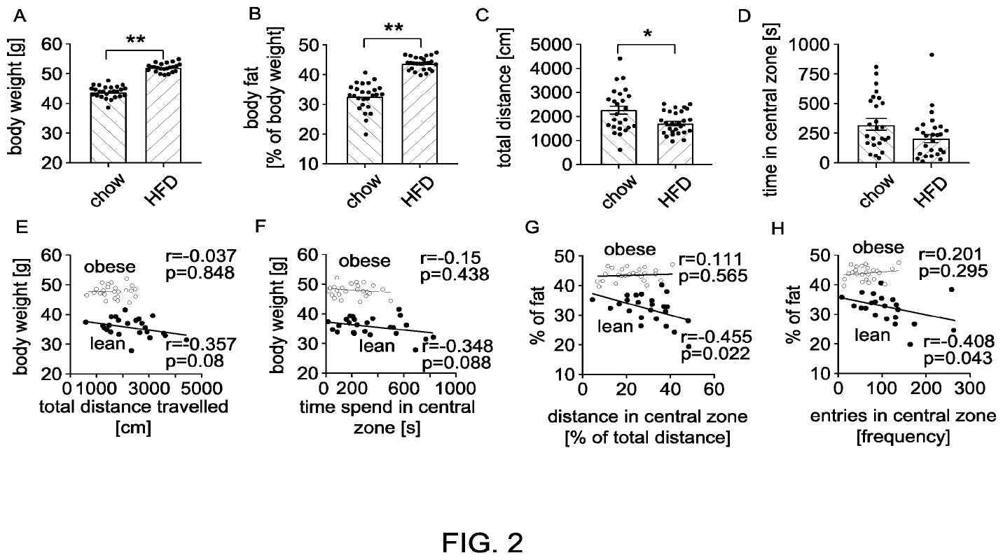

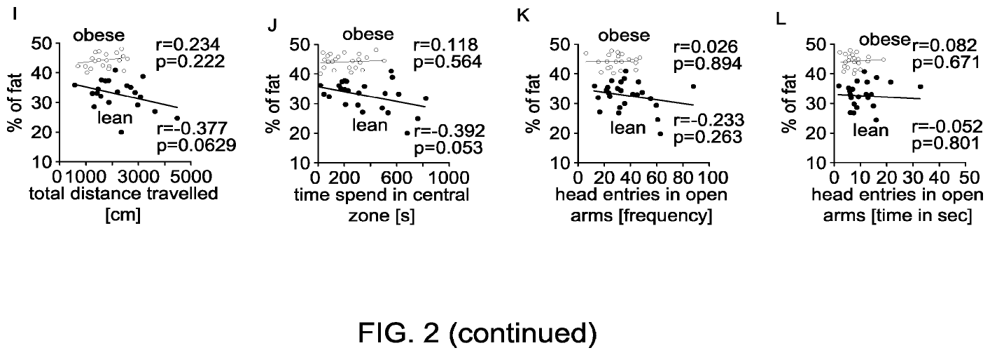

In order to investigate the relationship between obesity and anxiety, eight month old C57Bl/6 mice were fed a high fat (60% of calories from fat) or standard chow diet for 2 months. It was found that body weight and body fat content were increased in high fat diet (HFD) mice in comparison to chow-fed controls (FIG. 2A, B). To measure anxiety-like behavior, the open-field (OF) test was employed. This test evaluates the tendency of mice to remain close to the walls and avoid open spaces (central zone), a phenomenon known as thigmotaxis, which is widely used as an indication of anxiety-like behavior (Simon et al., Behav Brain Res 61:59-64 (1994)). HFD fed mice were less inclined to explore the central area of the open-field test chamber than the peripheral zone (FIG. 1A-C; FIG. 2D) and likewise the total distance covered was significantly decreased in HFD animals during the test (FIG. 2C). In order to account for the decreased activity in obese animals, anxiety measurements were analyzed as a function of the total distance travelled during experimental testing (FIG. 1B, D). It was next investigated if body weight and body composition alone could explain the observed anxiety-like behavior. Linear regression analysis revealed no significant correlation between body weight or % of fat mass on anxiety-like behavior in HFD fed mice (FIG. 1D, E; FIG. 2E-J). This indicates that while weight gain is associated with the onset of anxiety-like behavior, if a certain weight is reached, no correlation between weight and anxiety is found, which suggests that other factors apart from weight gain must play a role.

As an additional measurement of anxiety-like behavior, the elevated plus maze (EPM) test was used. The EPM is based on the animal's natural fear of heights and open spaces. Increased anxiety-like behavior in the EPM test is manifested as a decrease in the number of head-pokes and entries into the open arms. It was found that animals on a HFD had decreased entries into the open arms of the EPM (frequency and time) compared to lean animals (FIG. 1F-H), which is indicative of an increased anxiety-like behavior. Similarly to the OF test, there were no significant correlations between body and fat mass and anxiety parameters in both lean and HFD animals (FIG. 1I, J; FIG. 2G, H).

### Pharmacogenetic and Pharmacologic Clearance of Senescent Cells Alleviates Obesity-Related Behavioral Changes

It was investigated if senescent cells could contribute to anxiety-like behavior during obesity by using the INK-ATTAC mouse model, which allows the induction of suicide gene-mediated ablation of p16^{Ink4a}-expressing senescent cells upon administration of the drug AP20187 (AP) (Baker et al., Nature 479:232-236 (2011); Xu et al., Elife 4:e12997 (2015)).

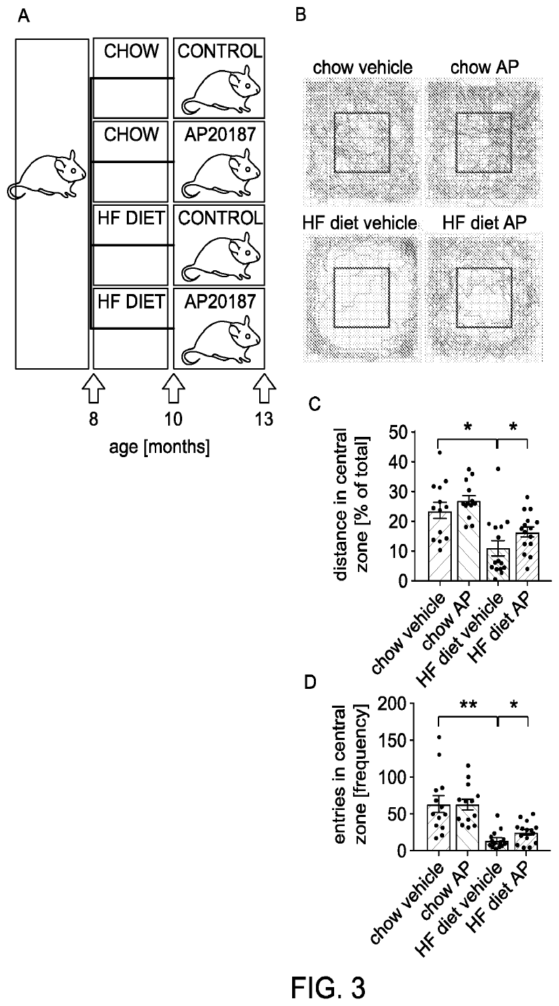

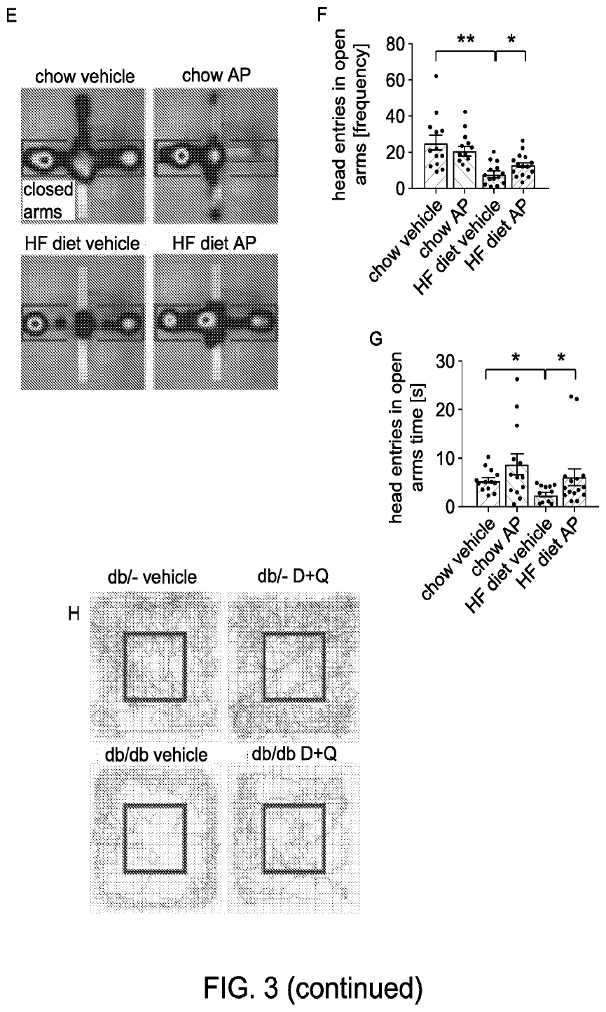

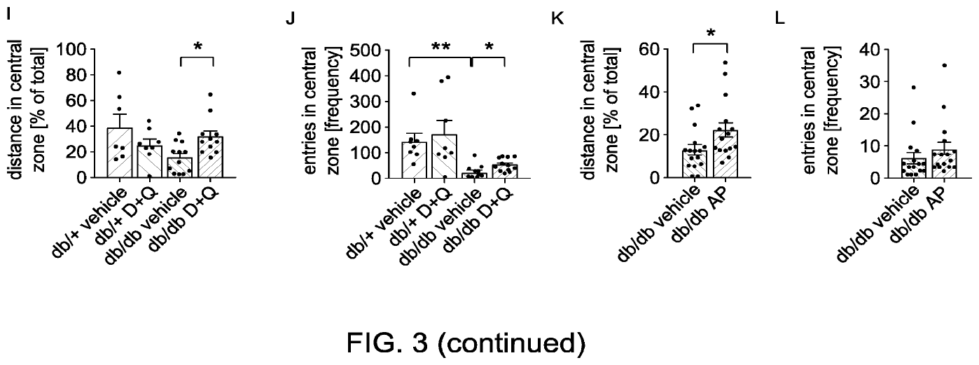

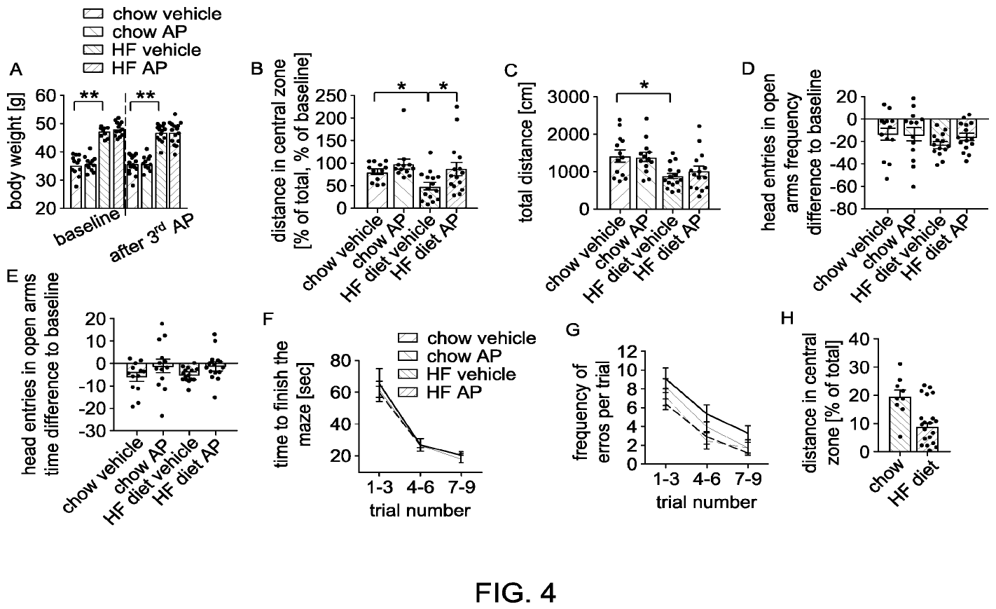

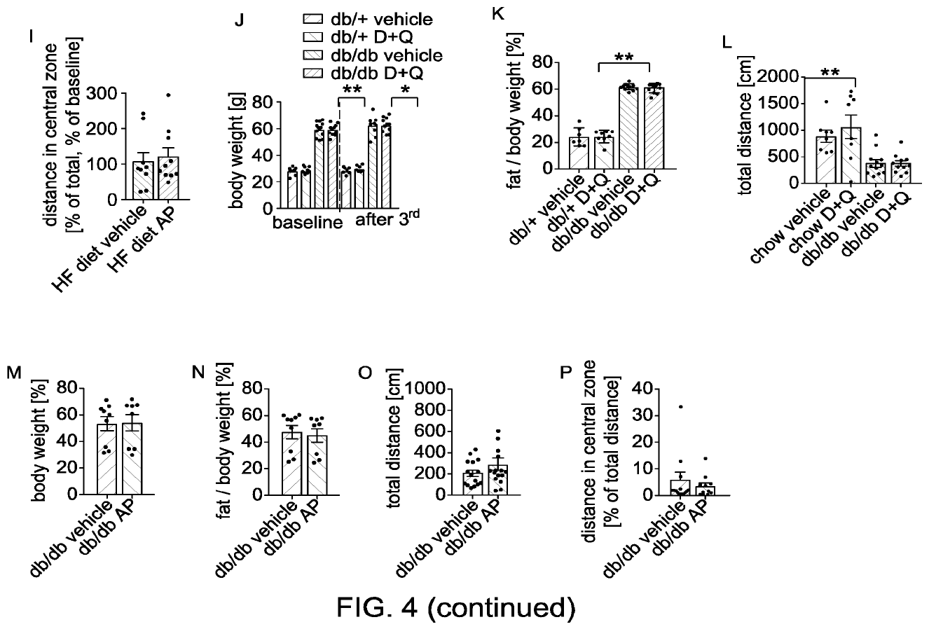

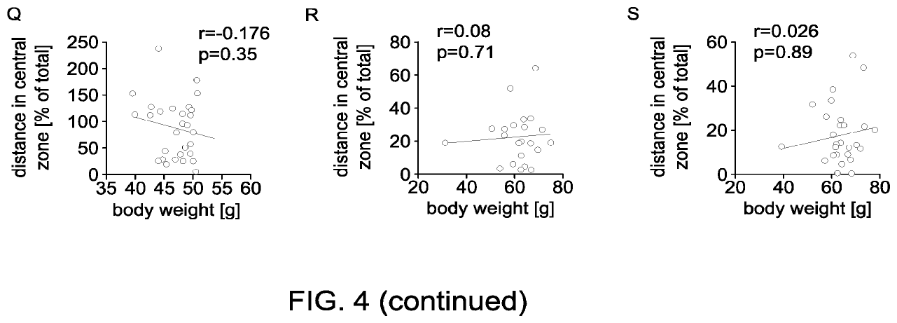

Chow- and HFD-fed 10 month old mice were repeatedly treated with AP or vehicle (FIG. 3A) over the duration of 10 weeks, which resulted in no significant changes in body weight (FIG. 4A) or body composition (not shown). To measure anxiety-like behavior, the OF test was used and a previous observation that animals on HFD were less inclined to explore the center of the open field chamber than the periphery as measured by distance travelled (FIG. 3C) and entries (FIG. 3D) to the central zone was confirmed. Furthermore, mice on HFD travelled significantly less throughout the duration of the tests, covering a smaller total distance (FIG. 4C). To take this into account, all measured parameters were expressed as a function of the total distance travelled. Clearance of p16^{Ink4a}-positive cells with AP reduced HFD-induced anxiety-like behavior as measured by distance covered in the central zone (FIG. 3B, FIG. 4B) and entries into the central zone (FIG. 3C). However, AP treatment did not affect the total distance travelled (FIG. 4C) or any of aforementioned parameters in chow-fed mice (FIG. 3B-D).

Next, anxiety-like behavior was assayed with the elevated plus maze (EPM). As previously observed, obese animals avoided entries into the open arms of the EPM (frequency and time) compared to lean animals (FIG. 3E-G and FIG. 4D, E), indicating increased anxiety-like behavior. Consistent with the data from the OF test, AP treatment significantly decreased the anxiety-like phenotype in obese animals as indicated by the frequency of head entries into the open arms of the EPM (FIG. 3E-G and FIG. 4D, E). Other cognitive functions, such as short-term memory (FIG. 4F, G), measured by the Stones maze test, were not altered by HFD or AP treatment. Altogether, these results show that specific elimination of p16^{Ink4a+}-senescent cells from obese INK-ATTAC mice alleviates anxiety-like behavior, but has no effect on memory performance.

To exclude off-target effects of the drug AP, wild-type C57Bl/6 mice were treated with the drug and tested for anxiety-like behavior. Wild-type mice showed a significant difference between chow and HFD in the OF test before the start of the treatment (FIG. 4H), but no difference was observed in HFD fed mice after treatment with AP (FIG. 4I).

In addition to HFD fed mice, complementary experiments were conducted in db/db mice in which obesity is caused by a point mutation in the leptin receptor gene lepr, leading to spontaneous type 2 diabetes (Wang et al., Current Diabetes Reviews 10:131-145 (2014)). These mice were treated intermittently for two months with the senolytic drug cocktail, Dasatinib and Quercetin (D+Q) (Zhu et al., Aging Cell 14:644-658 (2015)). Db/db mice have significantly increased body weights and adipose depot weights when compared to lean db^{+/-} heterozygous littermates, but interestingly body weight did not change over the course of D+Q treatment (FIG. 4J, K).

Similarly to HFD fed mice, db/db mice exhibited increased anxiety-like behavior as assessed by the OF test (FIG. 3H-J). It was observed that the total distance covered (FIG. 3I) and the number of entries (FIG. 3J) into the central zone were significantly reduced in db/db mice compared to their non-obese, db^{+/-} heterozygous littermates, a phenotype which could be significantly alleviated by treatment with senolytic compounds Dasatinib and Quercetin (D+Q). Obese db/db mice covered a significantly shorter distance in comparison to lean littermates, however D+Q treatment did not change total distance covered in db/db or db/db^{+/-} mice (FIG. 4L).

Finally, these results were confirmed in a cohort of double-transgenic, INK-ATTAC;db/db mice. Similarly to treatment with D+Q, genetic clearance of p16^{Ink4a}-positive senescent cells in INK-ATTAC;db/db mice did not alter body weight, body composition, or activity (FIG. 4M-O). Importantly, clearance of p16^{Ink4a}-positive senescent cells in INK-ATTAC;db/db mice significantly reduced anxiety-like behavior as assayed by OF test (FIG. 3K, L). To exclude off-target effects of the drug AP, db/db mice were treated with the drug. No off-target effects of the AP drug were observed on anxiety-like behavior by OF test (FIG. 4P). Linear regression analysis showed no significant correlations between body weight and anxiety-like behavior in HFD fed INK-ATTAC (with and without AP) or db/db (with or without AP or D+Q) (FIG. 4Q-S).

These data show that pharmacological or pharmacogenetic clearance of senescent cells in two different models of obesity significantly alleviates anxiety-like behavior.

### Pharmacological and Pharmacogenetic Senolytic Approaches Reduce Senescent Cell Burden and Alleviate Systemic Inflammation

To investigate the effectiveness of senescent cell-clearance in HFD mice, senescent markers were measured in the perigonadal adipose tissue, a tissue previously shown to exhibit a marked increase in the number senescent cells with age (Schafer et al., Nature Commun. 8:14532 (2017); Tchkonia et al., Aging Cell 9:667-684 (2010); Xu et al., Elife 4:e12997 (2015)).

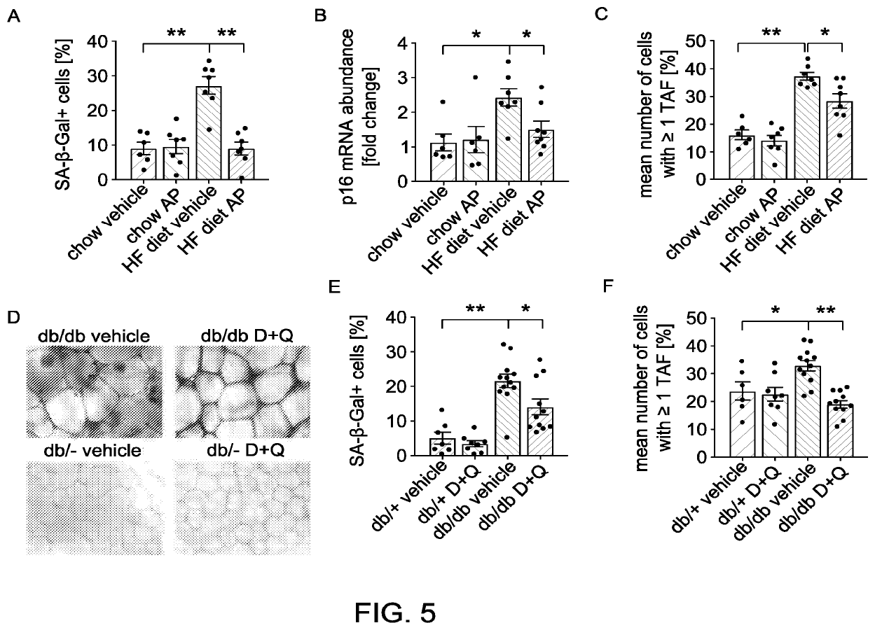

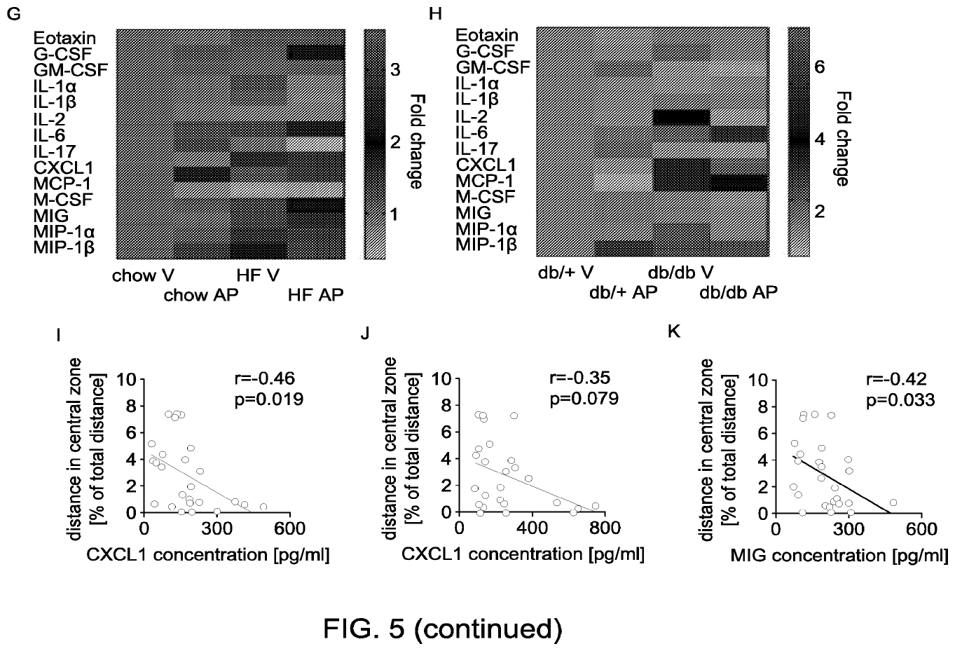

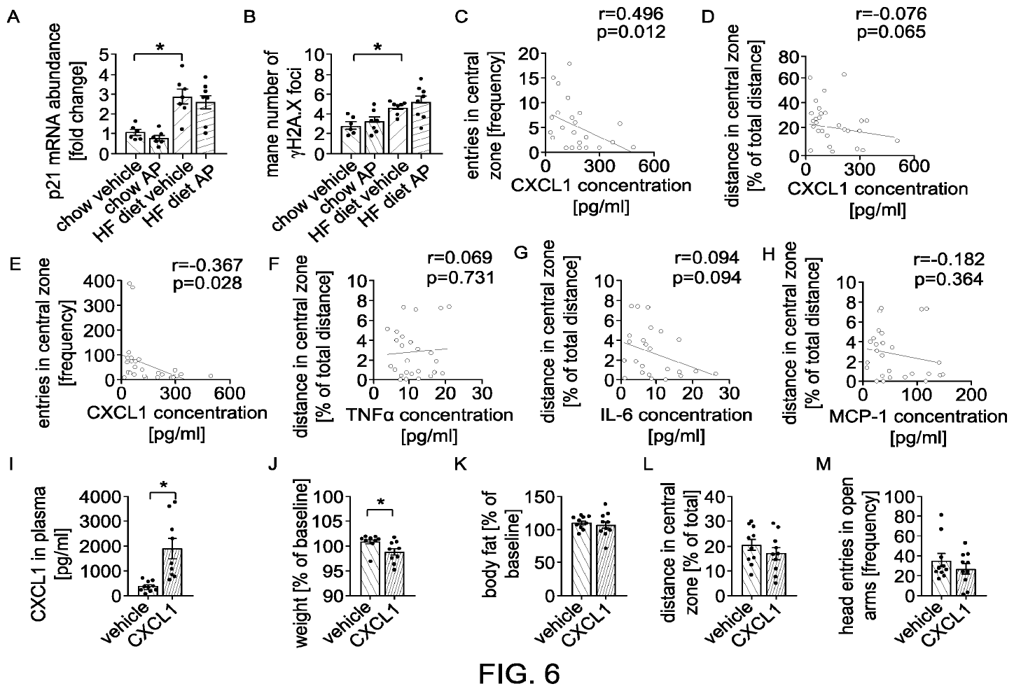

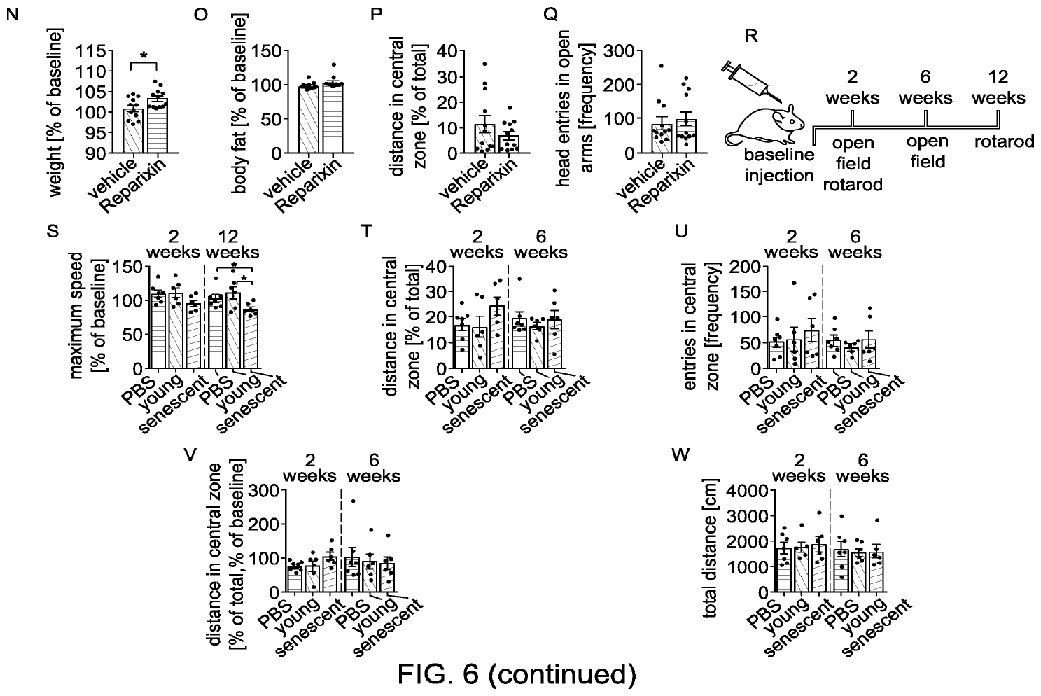

It was found that the senescence markers SA-β-Gal, p16^{Ink4a}, and telomere-associated DNA damage foci (TAF) were increased in INK-ATTAC mice on HFD and were significantly reduced upon administration of AP (FIG. 5A-C). p21 and γ-H2A.X were increased in HFD animals, but were not significantly changed upon AP treatment (FIG. 6A, B). Similarly, it was found that db/db mice had an increased senescent cell burden (measured by SA-β-Gal and TAF frequency) compared to db/db′ mice in the perigonadal fat, which was significantly reduced by treatment with the senolytic cocktail D+Q (FIG. 5D-F).

Given that the senolytic approaches applied act systemically, it is possible that they reduce SASP factors which can penetrate the blood-brain-barrier and therefore impact on the brain. To investigate that, blood plasma was analyzed for a large array of SASP factors.

Evaluation of circulating cytokines in the bloodstream of chow- and HFD-fed INK-ATTAC mice revealed that HFD resulted in the up-regulation of known SASP factors such as G-Csf, Il-1β, Kc/Cxcl1, Mcp-1, Mig, and Tnf-α which were down-regulated upon AP treatment (FIG. 5G). Similarly, SASP factors in the blood plasma of db/db mice were increased in comparison to lean db/+ littermates and reduced after treatment with D+Q (FIG. 5H). The expression of cytokines in the bloodstream was correlated with parameters of anxiety-like behavior measured in OF test. Significant negative correlations were observed between the plasma levels of Cxcl1, G-Csf, and Mig and different anxiety-like measurements in AP-treated HF mice (FIG. 5I-K and FIG. 6C) and D+Q-treated db/db mice (FIG. 6D, E), whereas no correlation was found for Tnf-α, Il-6, and Mcp-1 (FIG. 6F-H). These results led to further investigation of the impact of systemic factors on the observed anxiety phenotype. Lean animals were injected with Cxcl1. Injection of Cxcl1 lead to increased levels of circulating Cxcl1 (FIG. 6I). Additionally, increased plasma Cxcl1 decreased slightly the body weight, but did not change body composition (FIG. 6J, K). Examination of anxiety-like behavior using the OF (FIG. 6L) and EPM (FIG. 6M) tests showed no difference between treated and non-treated animals. To further examine the role of Cxcl1 in anxiety-like behavior, HFD mice were treated with Reparixin, which inhibits Cxcl1 receptors Cxcr1 and Cxcr2. Mice on Reparixin showed a small increase in body weight (FIG. 6N) but no difference in body fat (FIG. 6O) in comparison to non-treated mice. Again no difference was observed in behavior between the treated and non-treated groups, when animals were tested in the OF box (FIG. 6P) or the EPM (FIG. 6Q). These results suggest that Cxcl1 alone is not sufficient to induce an anxiety-like phenotype, however, it does not exclude the possibility that other soluble SASP factors are involved in the process.

Recently, it has been shown that transplantation of relatively low numbers of senescent cells in young animals resulted in physical dysfunction measured by Rotarod performance, grip strength, or endurance when compared to transplantation of young cells (Xu et al., Nature Medicine 24:1246-1256 (2018)). This study showed that transplantation of senescent cells resulted in long-lasting systemic effects in tissues located distantly from where senescent cells were injected.

To test if senescent cells could induce anxiety-like behavior via systemic effects, young or senescent cells were transplanted into lean mice and assessed behavior and physical function 6 and 12 weeks later. It was confirmed the previous observations that transplanted senescent cells reduced physical function, as measured by Rotarod (FIG. 6J), but had no effect on anxiety-like behavior using the OF test (FIG. 6T-W). Together, these experiments suggest that presence of senescent cells elsewhere in the body are not sufficient to induce an anxiety-like phenotype in mice.

### Senolytic Treatment Reduces the Frequency of Senescent Cells in Amygdala and Hypothalamus but not Other Regions of the Brain

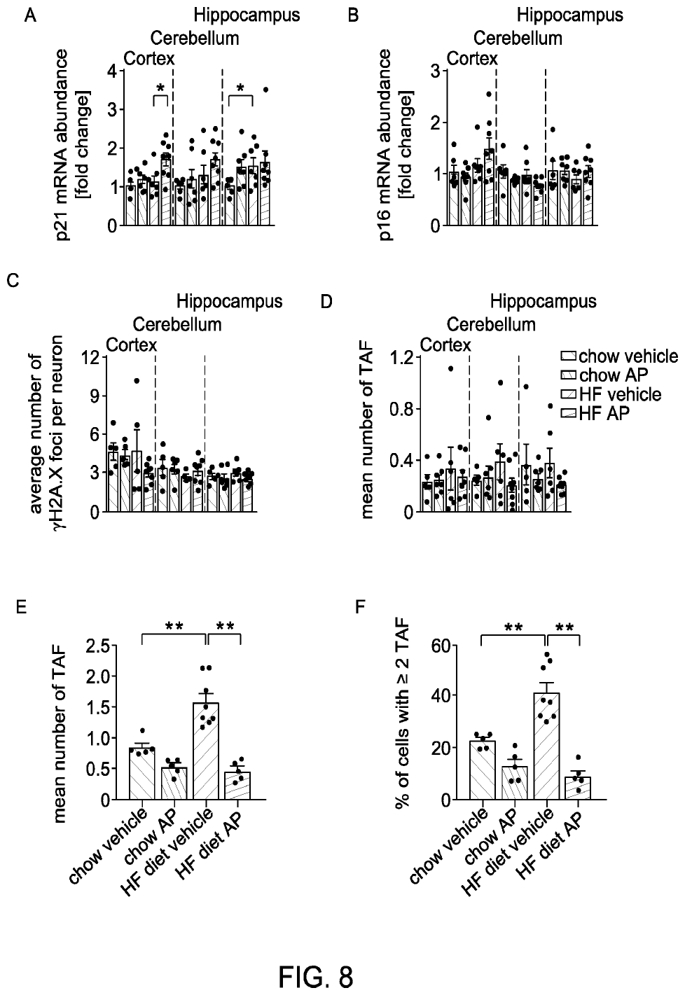

It was next examined if obesity could induce senescence specifically in the brain, thereby contributing to anxiety. Markers of senescence were first assessed in these regions of the brains of obese and lean INK-ATTAC mice treated with and without AP. No differences were found in the senescent markers p21, p16, γ-H2A.X, and TAF between any of the experimental groups (FIG. 8A-D). This correlates with the absence of differences in memory and learning in any of the experimental groups as assessed by the Stone's maze (FIG. 4F, G).

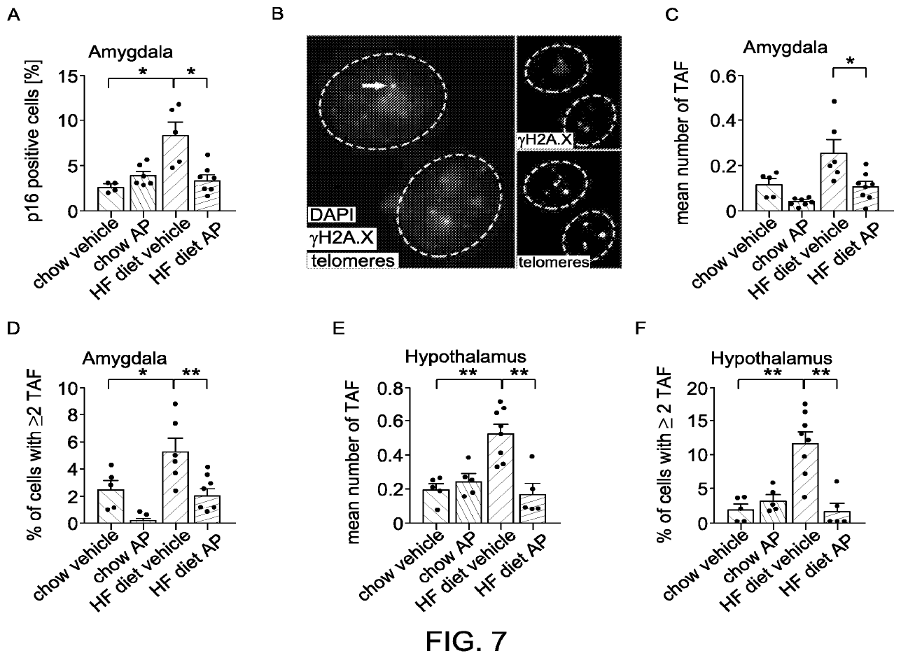

Interestingly, assessment of senescent cells in the amygdala, a brain region associated with emotional responses including anxiety and fear (Adhikari et al., Nature 527:179-185 (2015)), exhibited a significant increase in the number of p16^{Ink4a}-positive cells in HFD-fed mice (FIG. 7A). Analysis of TAF positive neurons in the basomedial layer of the amygdala showed a significant increase in HFD mice and a significant decrease after treatment with AP (FIG. 7A-D). Next, the abundance of senescent cells in the hypothalamus close to the 3^{rd} ventricle was analyzed and it was found that NeuN^{neg} (FIG. 7E, F) and NeuN^{pos} (FIG. 8E, F) cells show a significant increase in HFD mice compared to lean mice and a significant decrease after treatment with AP.

Together, these data indicate that HFD does not induce senescence in regions of the brain implicated in learning, memory, and motor-neuron control such as the cortex, cerebellum, and hippocampus. However, HFD induces senescence in the hypothalamus and amygdala, which may contribute to its effects on anxiety-like behavior and treatment with AP reduced senescent cell abundance and attenuated these behavioral changes.

### Clearance of Senescent Cells Decreases Periventricular Accumulation of Lipid-Laden Glia in Obese Animals

A connection between senescence and fat accumulating in the brain was investigated.

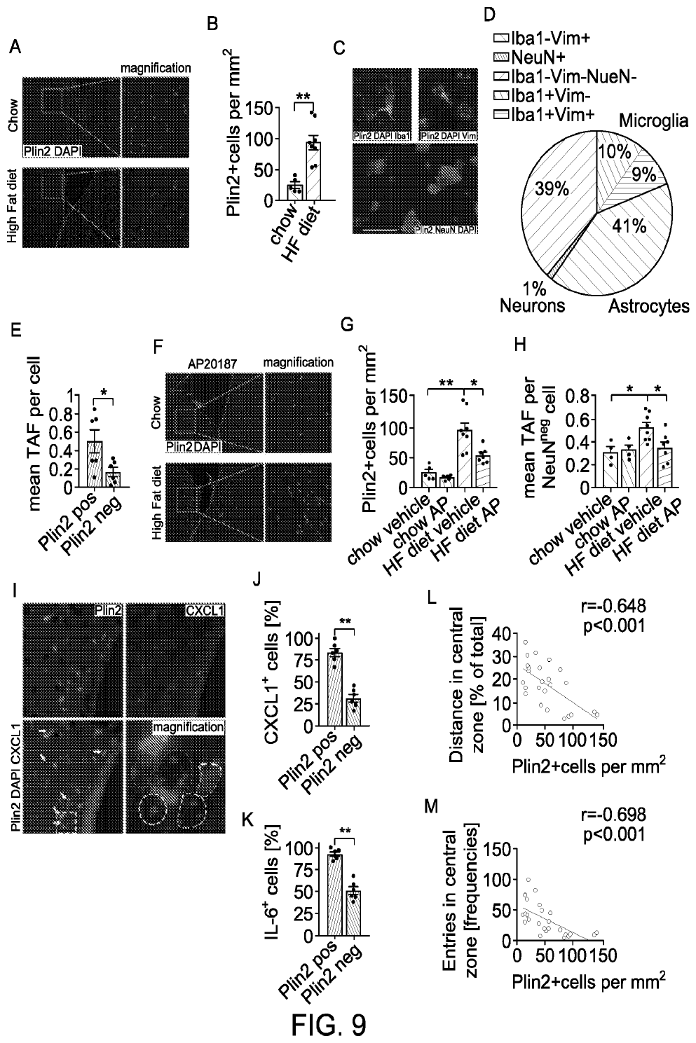

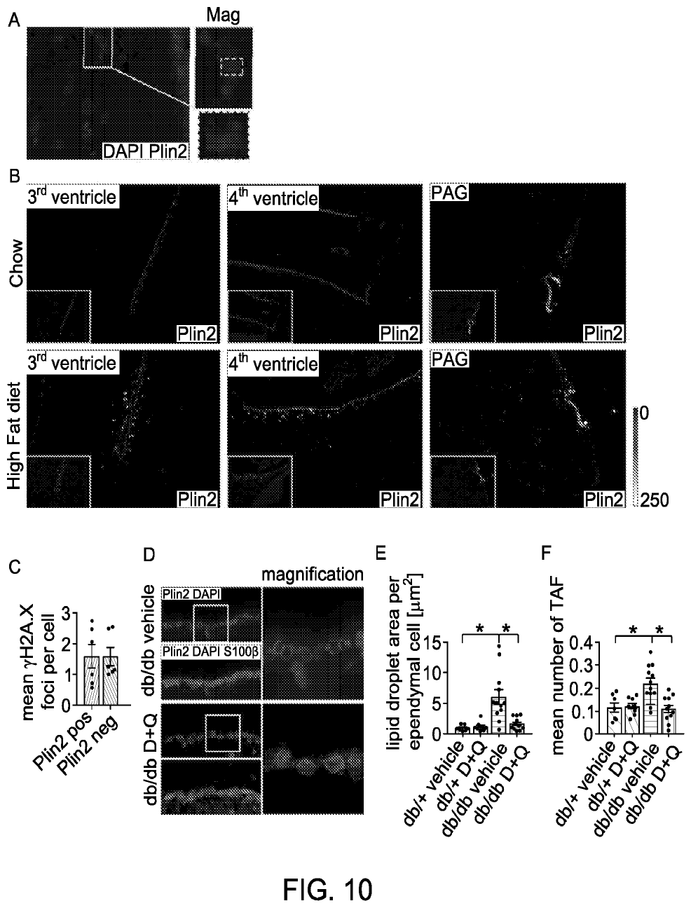

Analysis of Perilipin 2 (Plin2) expression (a protein which surrounds lipid droplets) in the brain of HF diet mice revealed a significant increase in Plin2⁺ cells (FIG. 10A) located in close proximity to the lateral ventricle (LV) compared to chow-fed controls (FIGS. 9A, B). Plin2⁺ cells were also detected around the 3^{rd} and 4^{th} ventricles and the periaqueductal gray (PAG) matter (FIG. 10B) but not in other brain regions. Double-staining for Plin2 and the cell type-specific markers, vimentin (Vim), Iba1, and NeuN, indicated that Plin2⁺ cells are mostly astrocytes (41%) and microglia (19%) (FIG. 9C, D).

In order to investigate if Plin2⁺ cells show features of senescence, the senescence marker TAF was analyzed in combination with immunostaining against Plin2. Higher mean values and higher frequencies of TAF in Plin2⁺ cells (FIG. 9E) were found, while total DNA damage did not change (FIG. 10C). Together, these findings indicate that a HFD contributes to increased numbers of senescent cells in the periventricular region of the brain and that these cells preferentially accumulate fat. Given that a similar phenomenon was observed in senescent hepatocytes and fibroblasts (see, e.g., Ogrodnik et al., Nat Commun. 8:15691 (2017)), this phenotype was termed Accumulation of Lipids in Senescence (ALISE).

To further investigate the impact of senescent cells on the build-up of fat in the brain, the INK-ATTAC mouse model (Baker et al., Elife. 4:e12997 (2015); and Baker et al., Nature 479:232-236 (2011)) was used. Treatment of HFD INK-ATTAC mice with AP resulted in a significant reduction of Plin2⁺ cells (FIG. 9F, G), as well as cells bearing senescent markers (FIG. 9H). Further, the frequency of cells containing lipid droplets and the senescent marker TAF in the LV of db/db animals was analyzed, and it was found that these were significantly increased in db/db in comparison to db/db^{-/+} animals and significantly reduced after treatment with the senolytic cocktail D+Q (FIG. 10D-F). Additionally, neuroinflammation was assessed by conducting RNA-In situ hybridization against SASP factors Cxcl1 and Il-6 in combination with immunostaining for Plin2 in the LV of HFD mice (FIG. 9I). Interestingly, the majority of Plin2⁺ cells were also positive for Cxcl1 and Il-6 (FIG. 9J, K).

Lastly, anxiety markers in HFD animals, such as distance travelled in the central zone and entries into the central zone, showed a strong negative correlation with the abundance of Plin2⁺ cells detected in the lateral ventricle (FIG. 9J, K). Together, these data support a causal link between the accumulation of lipid-laden senescent glial cells in obese animals and anxiety-like behavior.

### Suppression of the ALISE Phenotype Reduces Accumulation of Cytosolic Chromatin Fragments (CCF) and the SASP

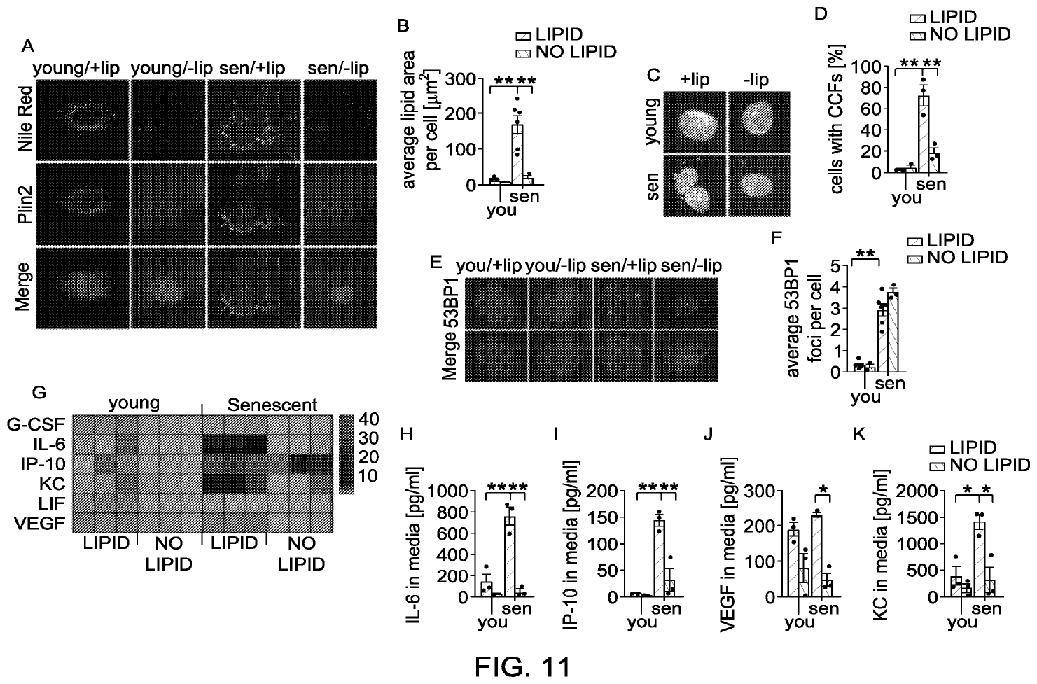

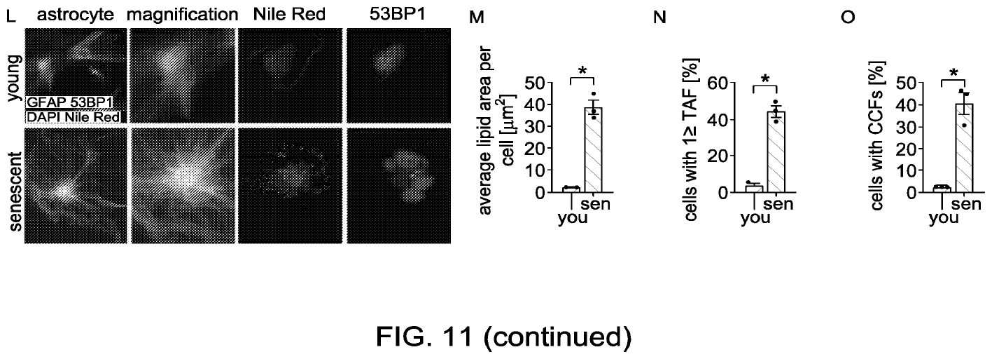

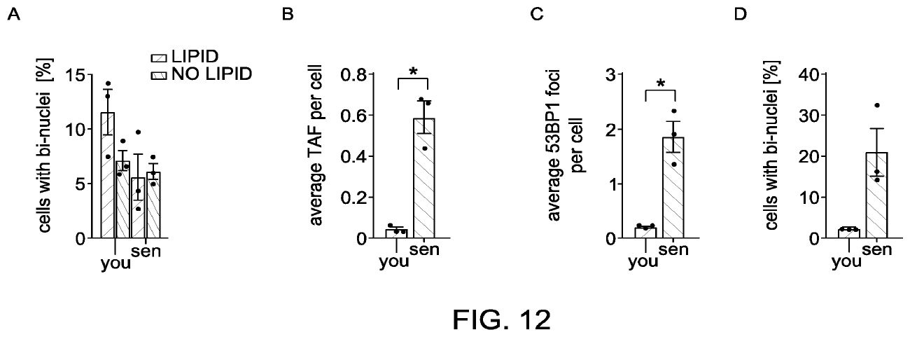

To further investigate the impact of fat accumulation on cell senescence, mouse adult fibroblasts (MAF) were used and senescence was induced by X-ray irradiation as described elsewhere (see, e.g., Jurk et al., Nat Commun 2 (2014); Ogrodnik et al., Nat Commun. 8:1569 (2017)). Senescent cells were cultured in the presence or absence of external sources of lipids. It was found that in the absence of extracellular lipids, the ALISE phenotype cells (assessed by lipophilic dye, Nile Red) was suppressed (FIG. 11A, B). Next, it was investigated if the impact of fat build-up on different markers of cellular senescence (FIG. 11C-K; FIG. 12A). Recently, it has been reported that senescent cells contain cytoplasmic chromatin fragments (CCF) (Ivanov et al., Journal of Cell Biology 202:129-143 (2013)), which activate the DNA-sensing cGAS-STING pathway which is a major driver of the SASP (Dou et al., Nature 550:402 (2017)). Interestingly, it was found that abrogation of the ALISE phenotype significantly reduced CCF in senescent cells (FIG. 11C, D), but not the average number of DNA damage foci (FIG. 11E, F) or bi-nuclearity (FIG. 12A). Consistent with the hypothesis that enhanced lipid deposition impacts on CCF and the SASP, it was found that depriving cells of lipids resulted in a drastic reduction of several key components of the SASP, such as Il-6, Kc (Cxcl-1), Ip-10 (Cxcl-10), and Lix (Cxcl-5) (FIG. 11G-K). To investigate if these findings were restricted to MAFs, similar experiments were conducted in primary mouse astrocytes. Similarly to MAFs increased build-up of fat, increased TAF, and higher numbers of CCFs were found in senescent astrocytes (FIG. 11L-O).

These data show that excessive lipid accumulation during senescence (ALISE) may be a contributor of genomic instability, resulting in release of chromatin fragments and activation of the SASP.

### Impaired Neurogenesis in HF Animals is Rescued by Clearance of Senescent Cells

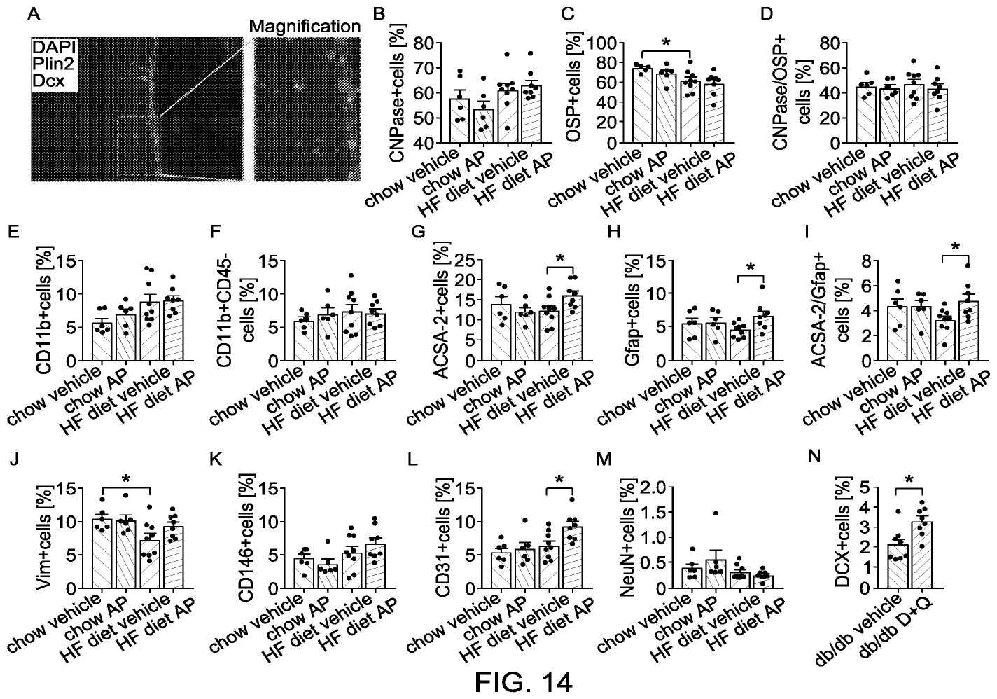

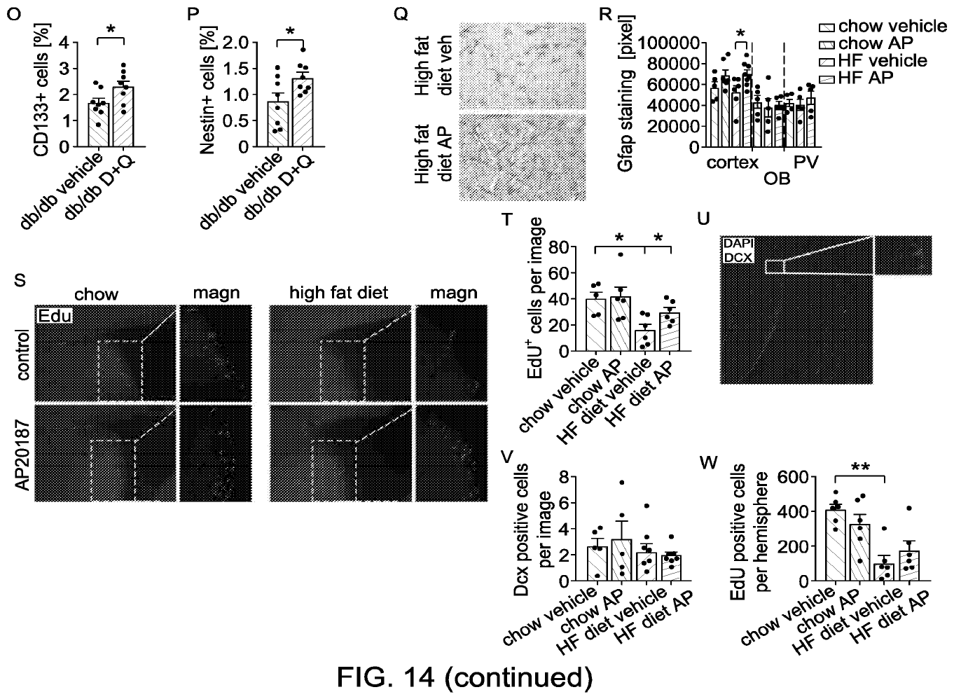

In HFD mice, it was observed that Plin2⁺ senescent glial cells are frequently found in close proximity to cells expressing doublecortin (Dcx), a marker of neuronal precursor cells and immature neurons (FIG. 14A). Thus, the presence of ALISE glial cells in the subventricular zone (SVZ) was investigated.

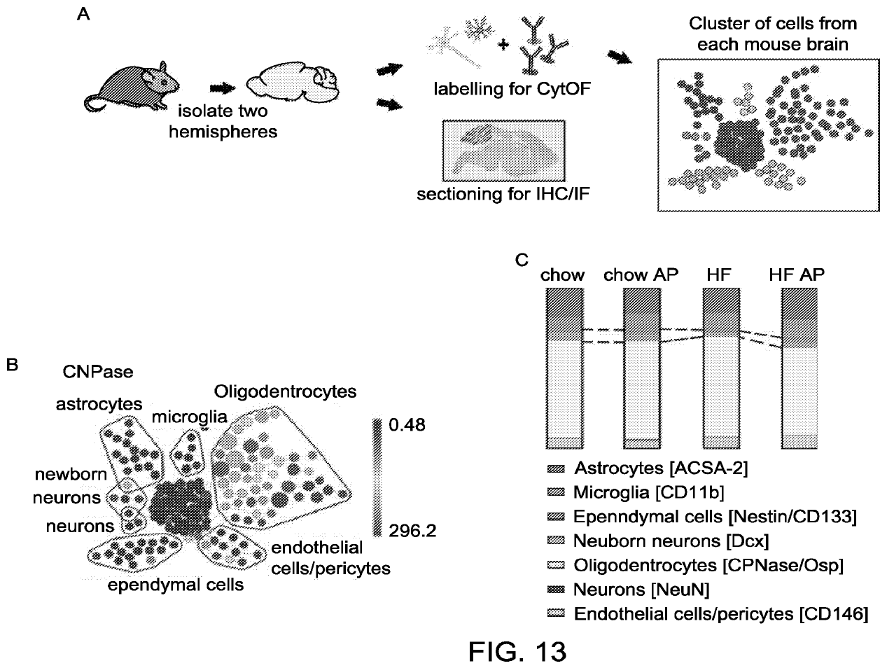

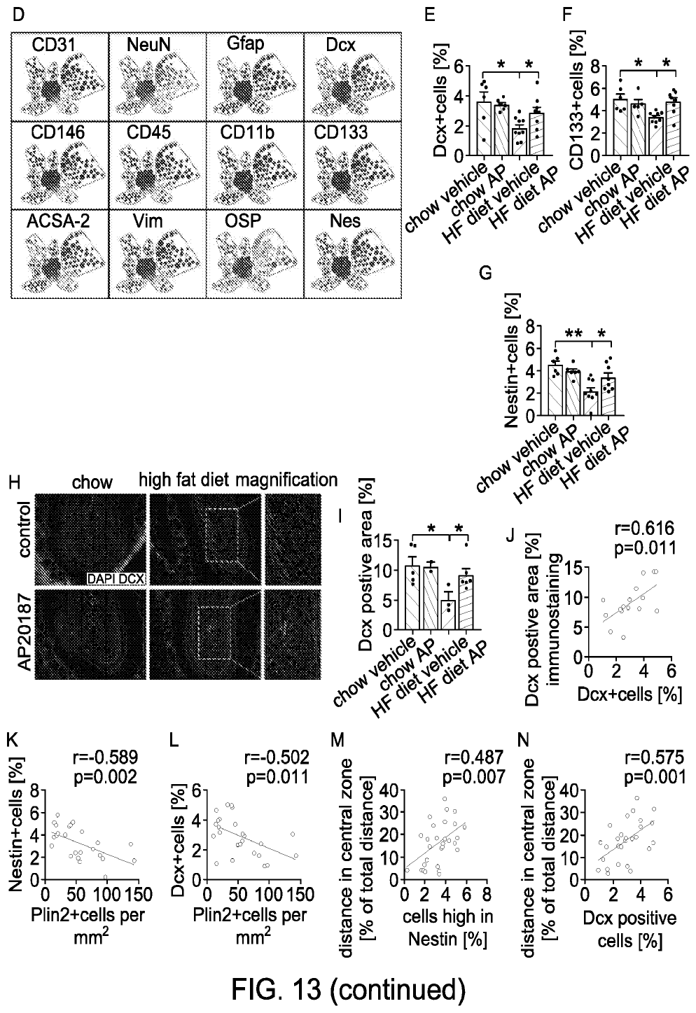

Lean and obese INK-ATTAC mice were treated with or without AP as previously described. Following organ harvesting, single-cell suspensions were obtained from one brain hemisphere and analyzed them by Cytometry by Time Of Flight (CyTOF), which allows mapping and discriminating between different brain cells including astrocytes, oligodendrocytes, microglia, neurons, ependymal cells, pericytes, and endothelial cells. The second brain hemisphere was reserved for histological analyses (FIG. 13A, B).

It was found that brains of mice fed a HFD did not exhibit significant changes in the frequencies of oligodendrocytes (CNPase⁺ or OSP⁺), microglia (CD11b⁺, CD45), mature neurons (NeuN⁺), or endothelial cells (CD31⁺ or CD146⁺) (FIG. 13C; FIG. 14B-M). However, populations of neuronal precursor cells (Nestin⁺), immature neurons (Dcx⁺), and ependymal cells (CD133⁺) were significantly decreased in animals subjected to HFD feeding (FIG. 13C-G). Clearance of senescent cells by AP did not alter the frequencies of oligodendrocytes (CNPase⁺ or OSP⁺), microglia (CD11b⁺, CD45⁻), mature neurons (NeuN⁺), or endothelial cells (CD146⁺) (FIG. 13C; FIG. 14B-M), but significantly increased neuronal precursor cells, immature neurons, and ependymal cells (FIG. 13C-G). AP treatment was sufficient to induce partial recovery of obesity-related stem cell depletion (FIG. 13C, E) and to replenish CD133⁺ and Nestin⁺ cell abundance (FIG. 13F, G). Analysis of cell populations in brains of db/db mice showed a similar pattern to HFD-fed mice. Markers for immature neurons (Dcx⁺), ependymal cells (CD133⁺), and neuronal precursor cells (Nestin⁺) were all significantly upregulated after senolytic treatment (FIG. 14N-P).

These findings were validated by performing immunostaining for Dcx (FIG. 13H, I) in the olfactory bulb and EdU-pulse labelling (FIG. 14S, T) in the subventricular zone of HFD animals. Analysis of Dcx⁺ cells in the olfactory bulb showed a positive correlation between the area occupied by Dcx⁺ cells in the granular cell layer of the olfactory bulb and the frequency of Dcx⁺ cells detected by CyTOF (FIG. 13J). Quantification of Dcx⁺ cells (FIG. 14U, V) and EdU⁺ cells (FIG. 14W) in the subgranular zone (SGZ) of the hippocampus did not correlate with results from CyTOF, implying that adult neurogenesis in SVZ, but not in the SGZ, is affected by the presence of senescent cells induced by obesity. Interestingly, clearance of these senescent cells led to a significant increase in the population of astrocytes (FIG. 14G-I) in obese animals, whereas no differences between lean and obese animals were detected. This finding was further confirmed by immunostaining for Gfap (FIG. 14Q, R). Finally, it was found that frequencies of Plin2⁺ glia correlated with markers of ependymal cells and markers of adult neurogenesis Nestin⁺ (FIG. 13K) and Dcx⁺ (FIG. 13L). Similarly, individual differences in distance travelled in the central area were positively correlated with frequencies of Dcx⁺ and Nestin⁺ cells (FIG. 13M, N).

In summary, these data indicate that senescent cells play a causal role in the decreased neurogenesis induced by HFD. Targeting senescent cells in obese mice alleviates obesity-related anxiety-like behavior related to clearance of periventricular fat accumulation and restoration of adult neurogenesis.

### Methods

### Animals

Experimental procedures were approved by the Institutional Animal Care and Use Committee at Mayo Clinic (protocol A26415). INK-ATTAC^{+/-} transgenic mice were generated and genotyped as described elsewhere (see, e.g., Baker et al., Nature 479:232-236 (2011)). Briefly, INK-ATTAC mice were produced and phenotyped at Mayo Clinic. Controls for the INK-ATTAC experiments were INK-ATTAC-null C57BL/6 background mice raised in parallel. C57BL/6 db/db and db/− mice were purchased from Jackson Laboratories.

Mice were housed 2-5 mice per cage, at 22+/−0.5° C. on a 12-12 hour day-night cycle and provided with food and water ad libitum. For high fat diet-induced obesity studies, mice were randomly assigned to chow or high fat diet groups. Mice were fed the high fat diet for 2-4 months before experiments started. High fat food was purchased from Research Diets (cat no #D12492). 60% of calories in this high-fat diet are from fat. Standard mouse chow diet was obtained from Lab Diet (cat no #5053).

INK-ATTAC mice were injected intraperitoneally (i.p.) with AP20187 (10 mg/kg) or vehicle for 3 days every 2 weeks for a total of 8-10 weeks.

Senolytic-treated db/db mice were gavaged with Dasatinib (D; 5 mg/kg) and quercetin (Q; 50 mg/kg) or vehicle for 5 days every 2 weeks for 8 weeks.

For off target effect measurements db/db and HDF mice (fed with HFD for 2 months prior treatment) were injected intraperitoneally (i.p.) with AP21087 at 10 mg/kg or vehicle for 3 days every 2 weeks for 8 weeks.

Recombinant CXCL1 (Peprotech, #250-11) or vehicle (PBS) was administered to lean C57BL/6 via i.p. injection (5 μg/kg in PBS) daily for 7 days. 2 hours after the last injection mice were tested in open field and elevated plus maze and afterwards dissected.

Reparixin L-lysine salt (MedChemExpress, #HY-15252) or L-Lysine hydrochloride (MedChemExpress, #HY-N0470) was dissolved in H₂O was administered to obese C57BL/6 mice (fed for 2 months with high-fat diet) via subcutaneous injection (30 mg/kg) twice per day for 2 weeks. 2 hours after the last injection mice were tested in open field and elevated plus maze and afterwards dissected.

Tissues from mice sacrificed at the indicated time points were snap-frozen in liquid nitrogen for biochemical studies or fixed in 4% PFA for 24 hours prior to processing and paraffin embedding. Paraffin-embedded tissues were cut at 3 μm or 10 μm intervals.

### Transplantation

Wild-type C57BL/6 mice were obtained from the National Institute on Aging (NIA) and maintained in a pathogen-free facility at 23-24° C. under a 12 hours light, 12 hours dark regimen with food and water ad libitum. Cell transplantation was done as previously described (Xu et al., Nature Medicine 24:1246-1256 (2018)). Briefly, when mice were 18 months of age, they were anesthetized using isoflurane and were injected intraperitoneally with 150 μl PBS through a 22-G needle, containing 10⁶ control or senescent mouse preadipocytes cells, or only PBS. Preadipocytes were obtained from inguinal fat from young Luciferase transgenic C57BL/6 mice from The Jackson Laboratory (Bar Harbor, Me.; stock no. 025854). Senescence was induced by 10 Gy of cesium radiation. Open field testing was carried out at 2 and 6 weeks after transplantation and Rotarod performance was tested 2 and 12 weeks after transplantation.

### Body Composition

Lean and fat mass of individual mice were determined by quantitative nuclear magnetic resonance using an EchoMRT analyser (Houston, Tex.) and expressed as a function of body weight. Un-anesthetized animals were placed in a plastic tube that was introduced into the EchoMRT instrument. Body composition, comprising fat mass and lean mass, was determined in approximately 90 seconds per animal.

### Open Field Testing

Locomotor activity and anxiety-like behavior of mice were assessed in sound-insulated, rectangular activity chambers (Med Associates Inc., St Albans, Vt., USA: W×L×D=27 cm×27 cm×20 cm with continually running fans, infrared lasers, and sensors). Beam breaks were assessed in 2-minute bins over 30 minutes, converted automatically to current mouse location and distance travelled (cm), and recorded on a computer with Med-PC software Version 4.0. Before the test, mice were acclimatized to the room for 1-1.5 hours before being introduced into the chambers. Mice were habituated for 5 minutes in the Open Field chamber (without recording) then placed for another 5 minutes in the home cage. Afterwards, mice were introduced back to the chambers and all mouse movements were recorded for 30 minutes. Anxiety was quantified by the distance mice travelled in the central 25% of the chamber (zone 1) as a function of the total distance mice travelled and by frequencies of entries into zone 1.

### Elevated Plus Maze

A grey colored elevated plus maze apparatus was used. Two open arms (25×5 cm) and two closed arms (25×5 cm) were attached at right angles to a central platform (5×5 cm). The apparatus was set 40 cm above the floor. Mice were first acclimatized to the room for 1-1.5 hours. Mice were then placed individually on the central platform with their back to one of the open arms. Before the test, mice were habituated for 1 minute to the maze, then placed back in the home cage for 5 minutes. Mice were tested for 5 minutes during which they could freely explore the apparatus. Tracking software (Ethovision) recognizes mouse head, central body point, and the base of the tail. Anxiety was quantified by frequency of and time spent during head pokes/dips toward open arms. Higher anxiety is indicated by a lower frequency of movement into open arms and less time spent there.

### Rotarod

Rotarod performance test evaluates mouse balance and motor coordination. Mice were brought to the test room a day before testing and habituated overnight. For the baseline tests, mice were trained on Rotarod (3375-M5; TSE systems) first for three consecutive days. Mice were placed (having their back turned towards the experimenter) on the rotating rod of 4.0 cm diameter. Mice trained to stay on the rod for 200 seconds at one constant speed per day, incrementing speed each day from 4 rpm, 6 rpm, and 8 rpm. If a mouse fell during training, it was put back on the rod. For the test on the fourth day, the Rotarod started at 4 rpm and steadily accelerates to 40 rpm over a 5 minute interval. The speed at which mice dropped was recorded, in four consecutive trials. 2 and 12 weeks after baseline measurements mice were tested again, habituating overnight prior test day. The average was normalized to the baseline and taken as an indicator of mouse balance and motor coordination.

### Stone's T-Maze

A water-motivated version of the Stone's T-maze was used to measure parameters of cognition. A straight run (for pre-training) or Stone's T-maze were placed into a steel pan filled with water to a depth of approximately 3 cm so that half the height of the interior walls of the maze were under water. The ceilings of both the straight run and maze were covered with clear acrylic to prevent mice from rearing out of the water. These dimensions created a situation that enables the mice to maintain contact with the floor while keeping their heads above water. The mice were placed into a start box and were pushed into the maze using a sliding panel. At the end of the straight run or maze there was a goal box that contains a ramp to a dry floor, which allows the mice to escape from the water upon successful completion of the straight run or maze. On day one, mice underwent straight run training to establish the concept that moving forward allows them to escape the water by reaching a water-free goal box. Successful completion of this phase requires the mice to reach the goal box in 10 seconds or faster in 8 out of 10 trials. Mice that did not reach this criterion were excluded from further testing. Maze training commenced the following day. Mice had to complete 9 maze acquisition trials in a single day. All mice per group performed one trial before performing the next one. Runs using between 6 and 8 mice resulted in inter-trial intervals (ITI) of approximately 5-12 minutes. During ITI, mice were placed in a holding cage containing a dry towel that was additionally heated by a red heat lamp. Primary measures of learning and memory were the latency to reach the goal box and the numbers of errors committed. An error was defined as complete entry of the mouse's head or the whole body into an incorrect path. During the acquisition phase, if any mouse failed to reach the goal box within 5 minutes, the trial was terminated and scored as a failure. Any mouse having 3 failures was removed from further trials. No mouse was excluded from this study.

### RT-PCR

Total RNA was extracted from white adipose tissue and brain using Trizol (Life Technologies, Carlsbad, Calif.) and reverse transcribed to cDNA with a M-MLV Reverse Transcriptase kit (Life Technologies). Real-time PCR was performed in a 7500 Fast Real Time PCR System (Applied Biosystems, Foster City, Calif.) using TaqMan Fast Universal PCR Master Mix (Life Technologies) and predesigned primers and probes from Applied Biosystems (Assay ID: Mm00494449_m1 [CDKN2A]; Mm04205640_g1 [CDKN1A]; Mm00446191_m1 [IL6]). Target gene expression was expressed as 2-ΔΔCT by the comparative CT method and normalized to the expression of TATA-binding protein (TBP) (Assay ID: Mm01277042 ml [TBP]).

### Cellular Senescence-Associated Beta-Galactosidase (SA-β-Gal) Activity

On the day of the sacrifice, a small piece of adipose tissue was fixed with 2% PFA and 0.5% glutaraldehyde (Sigma) for 15 minutes at room temperature before being incubated overnight in SA-β-Gal solution (150 mM NaCl (Sigma), 2 mM MgCl₂ (Sigma), 40 mM Citric Acid (Sigma), 12 mM NaPO₃ (Sigma), 400 μg/ml X-gal (Thermofisher), 2.1 mg/ml potassium hexacyanoferrat(II)trihydrate, and 1.65 mg/ml Potassium hexacyanoferrat(III)trihydrate (Sigma), pH 6.0) at 37° C. overnight. Fat chunks were washed with PBS three times and stored in PBS at 4° C. protected from light. Within 3 days, adipose tissue was stained with Hoechst solution (1:5000; Thermofisher), lightly squashed between two 1×3 inch glass slides, and imaged using a light microscope. 10-20 random visual fields were captured at 20× magnification at light exposure identical for all the samples. Images were quantified by manual counting of SA-β-Gal positive cells by a blinded assessor and the data were expressed as percent of total DAPI-positive cells.

### Mass Cytometry/CyTOF in Brain and Fat

This technique uniquely combines time-of-flight mass spectrometry with metal-labelling technology to enable detection of up to 40 protein targets per cell. A panel of antibodies based on surface markers, transcription factors, and cytokines (see Table 1) was designed for brain mass cytometry/cytometry by time of flight (CyTOF). Each antibody was tagged with a rare metal isotope and its function verified by mass cytometry according to the factory manual (Multi Metal labelling Kits, Fluidigm, CA). A CyTOF-2 mass cytometer (Fluidigm, South San Francisco, Calif.) was used for data acquisition. Acquired data were normalized based on normalization beads (Ce140, Eu151, Eu153, Ho165, and Lu175). A single brain hemisphere was dissociated into a single-cell suspension using brain tissue dissociation kits (Adult Brain Dissociation Kit, Miltenyi Biotec Inc., CA). Collected cells were incubated with metal-conjugated antibodies and, for testing intracellular proteins, including transcription factors and cytokines, fixation and permeabilization was conducted according to the manufacturer's instructions (Transcription Factor Staining Buffer Set, eBioscience, San Diego, Calif.). CyTOF data were analyzed by Cytobank (Santa Clara, Calif.).

TABLE 1
Antibodies used in CyTOF

| Name of an | Company producing | Catalogue | Metal | |
|---|---|---|---|---|
| antigen | antibody | number | Isotopes | Dilution |
| CD11b (Mac-1) | Fluidigm | 3154006B | 154Sm | 1:200 |
| CD45 | Fluidigm | 3089005B | 89Y | 1:400 |
| nestin | Abcam | ab6142 | 148Nd | 1:100 |
| Dex | Abcam | ab135349 | 173Yb | 1:100 |
| vimentin | Abcam | ab8978 | 161Dy | 1:100 |
| CD133 | Biolegend | 141202 | 153Eu | 1:100 |
| ACSA-2 | Miltenyi Biotec | 130099138 | 142Nd | 1:100 |
| Gfap | ebioscience | 14-9892-82 | 172Yb | 1:100 |
| CD31 | Fluidigm | 3165013B | 165Ho | 1:100 |
| NeuN | Abcam | ab177487 | 169Tm | 1:50 |
| CD146 | Fluidigm | 3141016B | 141Pr | 1:100 |
| CNPase | Abcam | ab53041 | 151Eu | 1:200 |
| OSP | Abcam | ab53041 | 159Tb | 1:200 |

### Cytokines

Serum levels of cytokines: Eotaxin, G-Csf, Tnf-α, Il-6, Ifn-γ, Il-1α, Il-1β, Il-17, Il-2, Kc/Cxcl1, Mcp-1, M-Csf, Mig, Mip-1α, and Mip-1β were determined using a Multiplexing LASER Bead Assay (Mouse Cytokine Array/Chemokine Array 31-Plex (MD31), Eve Technologies; Canada). Blood was withdrawn from mice by punctuation of the sub-mandibular vein at the day of dissection before an animal was sacrificed. 50 μL of serum were shipped to Eve Technologies on dry ice. Due to high variability of data an unbiased elimination of outliers was performed using ROUT's method (Graphpad 7 Prism). The same panel was used to detect SASP in MAF and 50 μl of media was shipped to Eve Technologies on dry ice.

### Cell Culture and Mouse Adult Fibroblasts (MAF)

MAF were extracted from 3-5 month old male C57BL/6 male mice. Ear clippings were transported and stored (not longer than 1 hour) in serum-free DMEM on ice. Punches were washed three times with serum-free media, finely cut, and incubated for 2-3 hours at 37° C. in DMEM containing 2 mg/ml collagenase A. A single-cell suspension was obtained by repeated pipetting and passing through a 24-G fine needle. Cells were centrifuged for 10 min at 1,000 r.p.m. and cultured in Advanced D-MEM/F-12 (DMEM, Invitrogen) plus 10% FBS (Sigma) in 3% 02 and 5% CO₂. Each cell strain was derived from a separate donor mouse and expanded until enough cells are generated for freezing aliquots. For each experiment, MAFs were defrosted, seeded and allowed to grow for 24 hours and then X-ray irradiated with 10 Gy using a PXI X-Rad 225 (RPS Services Ltd) to induce cellular senescence. Media were changed twice a week. The last medium change was performed at day 20 after senescence induction (IR) and cells were fixed in 2% PFA the next day.

For cytokine measurements media from the last 24 hours of culture (before cell fixation) were sent to Eve Technologies for SASP assessment (Mouse Cytokine Array/Chemokine Array 31-Plex (MD31)).

### Lipid Deprivation Experiments

Under normal conditions MAFs were kept in Advanced DMEM/F-12 (DMEM, Invitrogen) supplemented with 10% fetal bovine serum (FBS) (Sigma), 100 IU/ml penicillin/streptomycin, and 2 mM L-glutamine. In order to reduce content of lipids in tissue culture media, lipid-deprived FBS (Biowest) was used. Therefore, media containing standard FBS (with lipids) were designated “LIPID” and media containing lipid-deprived FBS were designated as “NO LIPID”. Young (control) cells were kept for at least 7 days under “NO LIPID” conditions before they were collected or senescence was induced.

### Mouse Neocortical Astrocytes

Astrocytes were extracted from 16-day-old embryo brains of either sex. At 16th day of pregnancy mice were sacrificed and brains of embryos were dissected. Neocortex was isolated and homogenized by pipetting through a fire-polished, FBS-coated Pasteur pipette. Bigger pieces of the neocortex were isolated by sedimentation and supernatant was centrifuged to isolate astrocytes. Astrocyte cultures were seeded at a density of 0.5×10⁶ cells/ml on culture dishes that had been coated previously with 15 μg/ml poly-1-ornithine overnight and subsequently washed with H₂O and PBS. Astrocytes were maintained in DMEM/F12 medium supplemented with 5 mM HEPES, 33 mM glucose, 13 mM sodium bicarbonate, 10% fetal bovine serum, 2 mM glutamine 100 U/ml penicillin and 100 μg/ml streptomycin (all from Invitrogen). Cells were cultured at 37° C. in a humidified atmosphere of 5% CO₂ and 3% 02. Induction of senescence and assessment of senescence markers and the ALISE phenotype were performed as for MAFs.

### Immuno- and Nile Red Staining for MAFs and Astrocytes

MAFs and astrocytes were plated on 19 mm (diameter) coverslips and at the end of experiments washed briefly with PBS and fixed for 10 minutes with 2% paraformaldehyde dissolved in PBS. Cells were permeabilized for 5 minutes with 0.5% TRITON X-100 dissolved in PBS. Cells were incubated with blocking buffer (5% normal goat serum (S-1000, Vector Laboratories) in PBS) for 60 minutes at room temperature. Plin2 (PROGEN #GP46, 1:250) 53BP1 (Novus Biologicals, #NB100-304, 1:250) and GFAP (Synaptic Systems, #173 004, 1:1000) antibodies were diluted in blocking buffer and applied overnight at 4° C. The next day, cells were washed three times with PBS and incubated for 60 minutes with secondary Alexa Fluor 594, goat, anti-guinea pig antibody (1:1000) for Plin 2 staining; Alexa Fluor, goat, anti-guinea pig (1:1000) for 53BP1 staining or Alexa Fluor, goat, anti-guinea pig (1:1000) for GFAP staining. For quantification of senescence markers coverslips were washed 3 times in PBS, then mounted in Vectashield, DAPI-containing mounting media. For assessment of lipid accumulation cells were washed 3 times with PBS before and after DAPI solution (PARTEC) was added for 30 minutes at room temperature. 2 μl of Nile red solution (Nile red (Sigma N3013) 150 μg ml⁻¹ in acetone) were added to 1 ml 80% glycerol (in Milli-Q water) and mixed thoroughly. 20 μl of Nile Red/glycerol were directly added to each cell sample and mounted on a glass microscope slide. Images were taken immediately after mounting using a Leica DM5500 widefield fluorescence microscope with a 20× objective lens. Area of lipid droplets was quantified using ImageJ (“Analyze particles” tool) in >50 cells in ≥10 images.

### EdU Experiments

For EdU experiments, HFD and control chow-fed INK-ATTAC mice treated with AP were injected with EdU (Life Technologies) at a dose of 123 mg/kg with the final concentration of 6.15 mg/mL, dissolved in sterile PBS (pH 7.4, Fisher Scientific), 2 hours before perfusion. 15 minutes intervals were allowed between mice injections to consider time needed for perfusing each mouse. The animals were deeply anesthetized with 90 mg/kg ketamine and 10 mg/kg of xylazine in sterile PBS prior perfusion. Transcardiac perfusion with PBS was followed by perfusion with 4% paraformaldehyde in PBS chilled on ice. Brains were harvested and postfixed overnight in 4% paraformaldehyde in PBS at 4° C., washed with PBS, and stored at 4° C. for vibratome sectioning. Sagittal brains sections of 50 μm were cut on a vibratome and collected sequentially in 6 different plate wells, total of 13 sections, 250 μm apart in each well, representing ⅙ of the brain hemisphere. Sections were stained free-floating in 12 well-plates, all procedures performed at room temperature at a volume of 500 μL for each well. Sections were initially permeabilized in 4% Triton X-100 (Sigma-Aldrich) in PBS for 1 hour with subsequent PBS washing for three times. Click reaction was performed for EdU visualization including 20 mM (+)-sodium L-ascorbate (Sigma-Aldrich), 10 μM Alexa 555-azide (Life Technologies), and 4 mM copper sulfate (Sigma-Aldrich) in PBS. Sections were incubated with gentle shaking for 15 minutes followed by PBS washing. Brain sections were collected and placed on gelatinized glass slides. All preparations were mounted with fluorescent mounting medium (DAKO) and coverslipped.

For assessment of hippocampal neurogenesis, EdU⁺ cells slides were imaged using a Leica DM5500B fluorescence microscope in depth Z stacking was used. Cells were manually counted in the basal layer of dentate gyrus in all 13 sections and multiplied by 6 to obtain as estimate of the number of dividing cells per hemisphere.

For assessment of neurogenesis in the subventricular zone (SVZ), 13 sagittal, 50 μm-thick sections were imaged using a Leica DM5500B fluorescence microscope. In depth Z stacking was used (images were captured as stacks separated by 4 μm with a 10× objective). Quantity of positive cells was manually counted in the ventral SVZ using ImageJ and the total number of cells was normalized to the number of images taken.

### Immunostaining and Telomere-Associated Foci (TAF) and Quantifications

Paraffin sections were deparaffinized with Histoclear and hydrated in an ethanol gradient followed by water and PBS. Antigen was retrieved by incubation in 0.01M citrate buffer (pH 6.0) at 95° C. for 10 minutes. Slides were placed in blocking buffer (1:60 normal goat serum [S-1000, Vector Laboratories] in 0.1% BSA/PBS) for 60 minutes at room temperature. For TAF staining, slides were additionally blocked with Avidin/Biotin (Vector Lab, #SP-2001) for 15 minutes each. Primary antibodies used (Table 2) were diluted in blocking buffer and applied overnight at 4° C. The next day, slides were washed 3 times with PBS and incubated for 30 minutes with secondary goat, anti-rabbit antibody (1:200; Vector Laboratories #BA-1000) for TAF staining or for 60 minutes with secondary Alexa antibody (Table 1). For TAF staining, Fluorescein-Avidin in PBS (1:500; #A-2011, Vector Lab) was applied to each sample for 20 minutes. Slides were washed 3 times in PBS, which was followed by FISH for TAF detection. Briefly, tissues were crosslinked with 4% paraformaldehyde for 20 minutes and dehydrated in graded ethanol. Sections were denatured for 10 minutes at 80° C. in hybridization buffer (70% formamide (Sigma), 25 mM MgCl₂, 0.1 M Tris (pH 7.2), and 5% blocking reagent [Roche]) containing 2.5 μg ml⁻¹ Cy-3-labelled telomere-specific (CCCTAA) peptide nucleic acid probe (Panagene), followed by hybridization for 2 hour at room temperature in the dark. Slides were washed twice with 70% formamide in 2×SSC for 15 minutes, followed by washes in 2×SSC and PBS for 10 minutes. Sections were mounted in Vectashield, DAPI-containing mounting media and imaged.

A single, 3 μm-thick section per mouse was used for TAF staining, while for Dcx and Plin2 staining was performed on three 10 μm-thick sections 80 μm-apart. To quantify periventricular lipid accumulation 10-30 images in the periventricular region were taken using the DM5500 widefield fluorescence microscope from Leica with a 10× (for frequency of periventricular glia) or ×40 (for ALISE phenotype of ependymal cells) objective lens. Number of Plin2-positive cells (for frequency of ALISE-positive periventricular glia) or area of Plin2-positive vesicles (for ALISE phenotype of ependymal cells) was assessed using ImageJ software. For identity assessment of Plin2+ cells, 10 μm-thick sections were stained with combination of antibodies for Iba1 (secondary antibody conjugated with Alexa Fluor 488), Plin2 (secondary antibody conjugated with Alexa Fluor 594), and Vimentin (secondary antibody conjugated with Alexa Fluor 647) and quantified for frequency of Plin2+ astrocytes (Iba1−, Vim+) and microglia (Iba1+) in the periventricular region. A separate staining for Plin2 (secondary antibody conjugated with Alexa Fluor 594) and NeuN (secondary antibody conjugated with Alexa Fluor 647) was used to determine frequency of Plin2+ neurons in periventricular region. For TAF quantification in depth Z stacking was used (images were captured as stacks separated by 0.4 μm with ×63 objective) followed by ImageJ analysis.

TABLE 2
Antibodies used in Immunostaining

| | Company producing | Primary | Secondary | Tertiary |
|---|---|---|---|---|
| | primary antibody | antibody: | antibody: | antibody or |
| | and catalogue | origin and | origin and | developing |
| | number | concentration | concentration | system |
| □-H2A.X | Cell Signalling, | Rabbit, 1:250 | Anti-rabbit, | DSC- |
| | #9718S | | biotinylated, | fluorescein |
| | | | Goat, 1:200 | (Vector Lab) |
| Perilipin 2 | Synaptic Systems, | Guinea pig, 1:250 | Anti-guinea pig, | |
| (Plin2) | #GP46 | | Alexa 594 or 488, | |
| | | | Goat, 1:1000 | |
| Iba1 | Abcam, | Goat, 1:250 | Anti-goat, | |
| | #ab5076 | | biotinylated, | |
| | | | donkey, 1:1000 | |
| S100β | Synaptic Systems, | Chicken, 1:1000 | Anti-guinea pig, | |
| | #287006 | | Alexa 594 or 647, | |
| | | | Goat, 1:1000 | |
| Vimentin | Abcam, | Chicken, 1:1000 | Anti-guinea pig, | |
| | #ab24525 | | Alexa 647, Goat, | |
| | | | 1:1000 | |
| Dcx | Cell Signalling | Rabbit, 1:250 | Anti-rabbit, | |
| (doublecortin) | #4604 | | Alexa 594, Goat, | |
| | | | 1:1000 | |
| NeuN/FoxO | Abcam | Mouse, 1:500 | Anti-mouse, | |
| | ab104224 | | Alexa 647, Goat, | |
| | | | 1:1000 | |
| Gfap | Synaptic systems, | Guinea pig, 1:500 | Anti-guinea pig, | |
| | #173004 | | Alexa 647, Goat, | |
| | | | 1:1000 | |
| EdU | Life technologies | | Alex555-azide, | |
| | #E10187 | | 10 μm | |
| | | | #A20012 | |

### IHC for GFAP

Tissue distribution glial fibrillary acidic protein (GFAP) in the brain was assessed by immunohistochemistry using an image analysis workstation after staining with antibodies. The brain sections were pretreated with 0.3% H₂O₂ methanol for 1 hour at room temperature and with normal goat serum for 1 hour at room temperature. Each specimen was incubated with the primary antibody overnight at 4° C. The primary antibody used in this study and the dilutions were as follows: rabbit anti-cow glial fibrillary acidic protein (GFAP) [1:800, DAKO, Denmark]. Immunohistochemistry was performed using the VECTASTAIN ABC System (Vector Laboratories Inc., Burlingame, Calif.) with the avidin/biotin peroxidase complex (ABC) method. Negative controls included replacement of the primary antibodies with normal rabbit serum [1:200, DAKO]. The immunoreactivity to rat positive control specimen of the primary antibodies was determined before use.

### RNA In Situ Hybridization

RNA-ISH was performed after RNAscope protocol from Advanced Cell Diagnostics Inc. (ACD). Paraffin sections were deparaffinized with Histoclear, rehydrated in graded ethanol (EtOH) and H₂O₂ was applied for 10 minutes at RT followed by two washes in H₂O. Sections were placed in hot retrieval reagent and heated for 15 minutes. After washes in H₂O and 100% EtOH sections were air dried. Sections were treated with protease plus for 30 minutes at 40° C., washed with H₂O and incubated with target probe (p16) for 2 hours at 40° C. Afterwards, slides were washed with H₂O followed by incubation with AMP1 (30 minutes at 40° C.) and next washed with wash buffer (WB) and AMP2 (15 minutes at 40° C.), WB and AMP3 (30 minutes at 40° C.), WB and AMP4 (15 minutes at 40° C.), WB and AMP5 (30 min at RT) and WB, and, finally, AMP6 (15 minutes at RT). RNAscope 2.5 HD Reagent kit-RED was used for chromogenic labelling. After counterstaining with haematoxylin, sections were mounted.

For analysis of cytokines (Il-6 and Cxcl1) sections were co-stained with antibodies for Plin2 and S100β. Briefly, following chromogenic labelling for cytokines, sections were washed 3 times in TBS for 5 minutes each followed by blocking in 0.1% BSA in PBS for 30 minutes at RT. Sections were incubated overnight with primary antibodies at 4° C. Next, sections were washes 3 times in TBS for 5 minutes each followed by secondary antibody incubation for 1 hour at RT. After 3 TBS washes sections were mounted using ProLong Gold mounting media containing DAPI. Probes used: Cdkn2a: 411011, Il-6: 315891, Cxcl1:407721 (all from ADC).

For all RNA-ISH experiments data was analyzed by quantifying the % of positive cells (which means each cell containing at least 1 focus was counted as positive).

### Statistical Analysis

Data are presented as mean±SEM for all data. All statistical analyses including testing the normality of data distribution were performed using GraphPad Prism 7.01 and a P value <0.05 was considered as significant. The study was designed to compare change in parameters between lean and obese animals and between obese and obese treated animals. All data were assessed for normality using D'Agostino & Pearson normality test (for n>7) or Shapiro-Wilk normality test (for n 7≥n>3). For 2-group comparisons and planned comparison 2-group comparisons (were used where appropriate when main effects were significant without significant interactions) data was further tested for equality of variances using F test. For non-normally distributed datasets (p<0.05 in D'Agostino & Pearson or Shapiro-Wilk normality tests) Mann-Whitney U test was used. For normally distributed datasets Welch's t-test (if p<0.05 in F test) or Student's t-test was used. For 2> groups comparisons one-way ANOVA with Tukey's multiple comparison test was used. For datasets split on two independent factors two-way ANOVA was used. Correlations were assessed using Pearson's (for datasets of normal distribution) or Spearman's (for datasets of non-normal distribution) rank correlation test.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for treating an obesity-induced neuropsychiatric disorder, wherein said method comprises administering a composition comprising a senolytic agent to a mammal identified as having an obesity-induced neuropsychiatric disorder.

2. The method of claim 1, wherein said mammal is a human.

3. The method of claim 1, wherein said senolytic agent is dasatinib or quercetin.

4. The method of claim 1, wherein said composition comprises dasatinib and quercetin.

5. The method of claim 1, wherein said obesity-induced neuropsychiatric disorder is obesity-induced anxiety.

6. The method of claim 1, wherein said obesity-induced neuropsychiatric disorder is obesity-induced depression.

7. The method of claim 1, wherein said composition is effective to clear senescent cells from within the brain of the mammal.

8. The method of claim 7, wherein said senescent cells comprise an accumulation of lipids in senescence phenotype.

9. The method of claim 7, wherein said senescent cells are cleared from in proximity to the lateral ventricle of the brain of the mammal.

10. The method of claim 1, wherein said composition is effective to decrease a level of one or more senescence-associated secretory phenotype (SASP) factor polypeptides in the mammal.

11. A method for increasing neurogenesis, wherein said method comprises administering a composition comprising a senolytic agent to a mammal identified as having an obesity-induced neuropsychiatric disorder under conditions wherein neurogenesis within said mammal is increased.

12. The method of claim 11, wherein said mammal is a human.

13. The method of claim 11, wherein said senolytic agent is dasatinib or quercetin.

14. The method of claim 11, wherein said composition comprises dasatinib and quercetin.

15. The method of claim 11, wherein said obesity-induced neuropsychiatric disorder is obesity-induced anxiety.

16. The method of claim 11, wherein said obesity-induced neuropsychiatric disorder is obesity-induced depression.

17. The method of claim 11, wherein said neurogenesis is increased in the brain of the mammal.

18. The method of claim 17, wherein said neurogenesis is increased in the subventricular zone of the brain of the mammal.

19. The method of claim 17, wherein said neurogenesis is increased in the olfactory bulbs of the mammal.

20. The method of claim 11, wherein said composition is effective to decrease a level of one or more senescence-associated secretory phenotype (SASP) factor polypeptides in the mammal.
